SEQUENCE LISTING (90 sequences)

SEQ ID NO: 1 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 2 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 3 - protein, 113 aa, synthetic construct
EVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 4 - protein, 113 aa, synthetic construct
EVQLVESGGGLVQPGGSLRLSCAASGFTFSNYGMSWVRQAPGKGLEWIGSIYYSGTTNYNPSLKSLVTISRDNSKNTLYLQMNSLRAEDTATYYCARESIDYWGQGTLVTVSS

SEQ ID NO: 5 - protein, 113 aa, synthetic construct
EVQLVESGGGLVQPGGSLRLSCAASGFTFSNYGMSWVRQAPGKGLEWIGSIYYSGDTNYNPSLKSLVTISRDNSKNTLYLQMNSLRAEDTATYYCARESIDYWGQGTLVTVSS

SEQ ID NO: 6 - protein, 113 aa, synthetic construct
EVQLVESGGGLVQPGGSLRLSCAASGFTFSNYGMSWVRQAPGKGLEWIGSIYYSGETNYNPSLKSLVTISRDNSKNTLYLQMNSLRAEDTATYYCARESIDYWGQGTLVTVSS

SEQ ID NO: 7 - protein, 113 aa, synthetic construct
EVQLVESGGGLVQPGGSLRLSCAASGFTFSNYGMSWVRQAPGKGLEWIGSIYYNGSTNYNPSLKSLVTISRDNSKNTLYLQMNSLRAEDTATYYCARESIDYWGQGTLVTVSS

SEQ ID NO: 8 - protein, 113 aa, synthetic construct
EVQLVESGGGLVQPGGSLRLSCAASGFTFSNYGMSWVRQAPGKGLEWIGSIYYDGSTNYNPSLKSLVTISRDNSKNTLYLQMNSLRAEDTATYYCARESIDYWGQGTLVTVSS

SEQ ID NO: 9 - protein, 113 aa, synthetic construct
EVQLVESGGGLVQPGGSLRLSCAASGFTFSNYGMSWVRQAPGKGLEWIGSIYYTGSTNYNPSLKSLVTISRDNSKNTLYLQMNSLRAEDTATYYCARESIDYWGQGTLVTVSS

SEQ ID NO: 10 - protein, 113 aa, synthetic construct
EVQLVESGGGLVQPGGSLRLSCAASGFTFSNYGMSWVRQAPGKGLEWIGSIYYSVSTNYNPSLKSLVTISRDNSKNTLYLQMNSLRAEDTATYYCARESIDYWGQGTLVTVSS

SEQ ID NO: 11 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 12 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 15 - protein, 583 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 16 - protein, 342 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 17 - protein, 583 aa, synthetic construct
EVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGTTNYNPSLKSLVTISRDNSKNTLYLQMNSLRAEDTATYYCARESIDYWGQGTLVTVSS

SEQ ID NO: 19 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGDTNYNPSLKSLVTISRDNSKNTLYLQMNSLRAEDTATYYCARESIDYWGQGTLVTVSS

SEQ ID NO: 20 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYNGSTNYNPSLKSLVTISRDNSKNTLYLQMNSLRAEDTATYYCARESIDYWGQGTLVTVSS

SEQ ID NO: 21 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 22 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYTGSTNYNPSLKSLVTISRDNSKNTLYLQMNSLRAEDTATYYCARESIDYWGQGTLVTVSS

SEQ ID NO: 23 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYNGQTNYNPSLKSLVTISRDNSKNTLYLQMNSLRAEDTATYYCARESIDYWGQGTLVTVSS

SEQ ID NO: 24 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYTGQTNYNPSLKSLVTISRDNSKNTLYLQMNSLRAEDTATYYCARESIDYWGQGTLVTVSS

SEQ ID NO: 25 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 28 - protein, 578 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 29 - protein, 453 aa, synthetic construct
EVQLVQSGGGVVRPGGSLRLSCAGSGFNFDDYGMSWVRQAPGKGLEWVSGINWNGGSTGYADSVKGRFTISRDNAKNSLYLQMNSLRAEDTALYYCARGAFWNSGSGNSPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, synthetic construct; identical to SEQ ID NO: 13

SEQ ID NO: 31 - protein, 581 aa, synthetic construct
EVQLVQSGGGVVRPGGSLRLSCAGSGFNFDDYGMSWVRQAPGKGLEWVSGINWNGGSTGYADSVKGRFTISRDNAKNSLYLQMNSLRAEDTALYYCARGAFWNSGSGNSPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSNYGMSWVRQAPGKGLEWIGSIYYSGQTNYNPSLKSLVTISRDNSKNTLYLQMNSLRAEDTATYYCARESIDYWGQGTLVTVSS

SEQ ID NO: 32 - protein, 630 aa, synthetic construct
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 33 - protein, 192 aa, synthetic construct
GMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQ

SEQ ID NO: 34 - protein, 184 aa, synthetic construct
LPKAVVKLDPPWIQVLKEDMVTLMCEGTHNPGNSSTQWFHNGRSVRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHT

SEQ ID NO: 35 - protein, 179 aa, synthetic construct
LQKAVVILDPEWVRVLEEDCVILRCQGTFSPEDNSTKWFHNKSLISHQDANYVIQSARVKDSGMYRCQTAFSALSDPVQLDVHADWLLLQTTKRLFQEGDPIRLRCHSWRNTPVFKVTYLQNGKGKKYFHRNSELSISKATHADSGSYFCRGIIGRNNISSASLQISIGDPTSPSSFLP

SEQ ID NO: 36 - protein, 184 aa, synthetic construct
GMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTIS

SEQ ID NO: 37 - protein, 450 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 38 - protein, 214 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 113 aa, synthetic construct
EVQLVESGGGLVQPGGSLRLSCAASGFTFSNYGMSWVRQAPGKGLEWIGSIYYNGQTNYNPSLKSLVTISRDNSKNTLYLQMNSLRAEDTATYYCARESIDYWGQGTLVTVSS

SEQ ID NO: 40 - protein, 113 aa, synthetic construct
EVQLVESGGGLVQPGGSLRLSCAASGFTFSNYGMSWVRQAPGKGLEWIGSIYYTGQTNYNPSLKSLVTISRDNSKNTLYLQMNSLRAEDTATYYCARESIDYWGQGTLVTVSS

SEQ ID NO: 41 - protein, 8 aa, synthetic construct
GFTFSNYG

SEQ ID NO: 42 - protein, 7 aa, synthetic construct
ARESIDY

SEQ ID NO: 43 - protein, 7 aa, synthetic construct
IYYSGQT

SEQ ID NO: 44 - protein, 7 aa, synthetic construct
IYYSGTT

SEQ ID NO: 45 - protein, 7 aa, synthetic construct
IYYSGDT

SEQ ID NO: 46 - protein, 7 aa, synthetic construct
IYYSGET

SEQ ID NO: 47 - protein, 7 aa, synthetic construct
IYYNGST

SEQ ID NO: 48 - protein, 7 aa, synthetic construct
IYYDGST

SEQ ID NO: 49 - protein, 7 aa, synthetic construct
IYYTGST

SEQ ID NO: 50 - protein, 7 aa, synthetic construct
IYYSVST

SEQ ID NO: 51 - protein, 7 aa, synthetic construct
IYYNGQT

SEQ ID NO: 52 - protein, 7 aa, synthetic construct
IYYTGQT

SEQ ID NO: 53 - protein, 5 aa, synthetic construct
NYGMS

SEQ ID NO: 54 - protein, 5 aa, synthetic construct
ESIDY

SEQ ID NO: 55 - protein, 16 aa, synthetic construct
SIYYSGQTNYNPSLKS

SEQ ID NO: 56 - protein, 16 aa, synthetic construct
SIYYSGTTNYNPSLKS

SEQ ID NO: 57 - protein, 16 aa, synthetic construct
SIYYSGDTNYNPSLKS

SEQ ID NO: 58 - protein, 16 aa, synthetic construct
SIYYSGETNYNPSLKS

SEQ ID NO: 59 - protein, 16 aa, synthetic construct
SIYYNGSTNYNPSLKS

SEQ ID NO: 60 - protein, 16 aa, synthetic construct
SIYYDGSTNYNPSLKS

SEQ ID NO: 61 - protein, 16 aa, synthetic construct
SIYYTGSTNYNPSLKS

SEQ ID NO: 62 - protein, 16 aa, synthetic construct
SIYYSVSTNYNPSLKS

SEQ ID NO: 63 - protein, 16 aa, synthetic construct
SIYYNGQTNYNPSLKS

SEQ ID NO: 64 - protein, 16 aa, synthetic construct
SIYYTGQTNYNPSLKS

SEQ ID NO: 65 - protein, 7 aa, synthetic construct
GFTFSNY

SEQ ID NO: 66 - protein, 5 aa, synthetic construct
ESIDY

SEQ ID NO: 67 - protein, 5 aa, synthetic construct
YYSGQ

SEQ ID NO: 68 - protein, 5 aa, synthetic construct
YYSGT

SEQ ID NO: 69 - protein, 5 aa, synthetic construct
YYSGD

SEQ ID NO: 70 - protein, 5 aa, synthetic construct
YYSGE

SEQ ID NO: 71 - protein, 5 aa, synthetic construct
YYNGS

SEQ ID NO: 72 - protein, 5 aa, synthetic construct
YYDGS

SEQ ID NO: 73 - protein, 5 aa, synthetic construct
YYTGS

SEQ ID NO: 74 - protein, 5 aa, synthetic construct
YYSVS

SEQ ID NO: 75 - protein, 5 aa, synthetic construct
YYNGQ

SEQ ID NO: 76 - protein, 5 aa, synthetic construct
YYTGQ

SEQ ID NO: 77 - protein, 6 aa, synthetic construct
SNYGMS

SEQ ID NO: 78 - protein, 6 aa, synthetic construct
ARESID

SEQ ID NO: 79 - protein, 12 aa, synthetic construct
WIGSIYYSGQTN

SEQ ID NO: 80 - protein, 12 aa, synthetic construct
WIGSIYYSGTTN

SEQ ID NO: 81 - protein, 12 aa, synthetic construct
WIGSIYYSGDTN

SEQ ID NO: 82 - protein, 12 aa, synthetic construct
WIGSIYYSGETN

SEQ ID NO: 83 - protein, 12 aa, synthetic construct
WIGSIYYNGSTN

SEQ ID NO: 84 - protein, 12 aa, synthetic construct
WIGSIYYDGSTN

SEQ ID NO: 85 - protein, 12 aa, synthetic construct
WIGSIYYTGSTN

SEQ ID NO: 86 - protein, 12 aa, synthetic construct
WIGSIYYSVSTN

SEQ ID NO: 87 - protein, 12 aa, synthetic construct
WIGSIYYNGQTN

SEQ ID NO: 88 - protein, 12 aa, synthetic construct
WIGSIYYTGQTN

SEQ ID NO: 89 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 23 aa, synthetic construct
EPKSCDKTHTCPPCPAPELLGGP

(19) United States
(12) Patent Application Publication
(10) Pub. No.: US 2026/0327314 A1
(43) Pub. Date: Oct. 22, 2026

(54) ANTI-CD16A ANTIBODIES AND USES THEREOF

(71) Applicant: SUZHOU BIOMISSILE PHARMACEUTICALS CO., LTD., Suzhou (CN)

(72) Inventors: Chao TU, Belmont, MA (US); Yi ZHANG, Suzhou (CN); Tianlei YING, Suzhou (CN)

(21) Appl. No.: 19/482,755

(22) PCT Filed: Mar. 27, 2024
(86) PCT No.: PCT/CN2024/084063

§ 371 Date: Nov. 9, 2025

(30) Foreign Application Priority Data
May 10, 2023 (WO) PCT/CN2023/093142

(51) Int. Cl. C07K 16/28; A61K 39/00; A61K 47/68; A61P 35/00; C07K 16/30; C07K 16/32

(52) U.S. Cl. CPC C07K 16/283; A61K 47/6849; A61P 35/00; C07K 16/30; C07K 16/32; A61K 2039/505; C07K 2317/31; C07K 2317/569; C07K 2317/92

(57) ABSTRACT

Disclosed is a single domain antibody specifically binding to CD16A, a heavy chain antibody comprising the single domain antibody, a bispecific antibody comprising the single domain antibody, an immunoconjugate comprising the single domain antibody, and a pharmaceutical composition and use thereof for treatment, prevention or diagnosis of cancers, acute or chronic infection, or aging-associated ailment.

### TECHNICAL FIELD

The present disclosure relates to anti-CD16A single domain antibodies, heavy chain antibodies, bispecific antibodies, immunoconjugates, pharmaceutical compositions and uses thereof in treating, preventing or diagnosing cancers, acute or chronic infection, or aging-associated ailment.

### BACKGROUND

Natural killer (NK) cells are defined by the expression of the cell adhesion marker CD56 and lack of the T-cell receptor CD3 (CD56+CD3). NK cells can be divided into 2 functionally distinct subsets, CD56^{bright} and CD56^{dim}, based on the cell surface density of CD56. Comprising approximately 10% of circulating NK cells, CD56^{bright} NK cells are generally thought to be more proliferative, to have a higher capacity for cytokine production after stimulation with IL-12 and IL-18, and to have poor cytotoxic effector activity at rest. CD56^{dim} NK cells, however, are potently cytotoxic without stimulation, mediate antibody dependent cellular cytotoxicity (ADCC), and produce cytokines after stimulation with target cells.

CD16 (FCγRIII) binds to the Fc portion of IgG antibodies; one type, CD16A, is a transmembrane protein that co-localizes with CD35 and Fc-εRI-γ on NK cells. Upon ligation, it induces a potent series of signals resulting in cytokine production and cytotoxic effector activity via ADCC. The second type, CD16B, is found on neutrophils and is not involved in tumor cell killing. Although the extracellular domains are highly homologous, glycosylphosphatidylinositol linkage differentiates CD16B from CD16A. Most CD56^{bright} NK cells in the peripheral blood express little to no CD16A. In contrast, the majority of CD56^{dim} cells uniformly express high levels of CD16A. Down-regulation of CD16A occurs after mitogen stimulation and coculture with malignant targets, an effect that is presumably mediated by a metalloprotease. This process may be important for rapid modulation of the surface density of CD16A, and in turn the activation status and effector function of NK cells.

The ADCC function of NK cells is highly pursued in antibody intervening immunotherapy. Bispecific antibodies with two targeting modules, one for recruiting ADCC receptors CD16A and the other for recognizing antigen, have drawn extensive attention due to the high efficacy for engaging NK cells to kill the target cells both in vitro and in vivo.

Currently, several formats of these bispecific antibodies are undergoing basic and clinical research, such as Bispecific Killer Cell Engager (BiKE), bispecific diabody (BidAb) and tetra-valent, bispecific TandAb. Recently, the more promising format TandAb has been developed by Affimed Therapeutics AG (Heidelberg, Germany). TandAb is a tetravalent, bispecific, tandem diabody composed of the tail-to-head homodimer of the two tandem scFv connected by three (GGS) 3 linkers. One TandAb, CD16/CD30 AFM13, has completed phase II clinical trials for patients with relapsed or refractory Hodgkin's lymphoma (clinical trial no. NCT02321592). However, TandAb likely will encounter more production problems than BidAb since it contains more sub-domains, all of which should be paired elegantly to promise a correct TandAb. Furthermore, due to its tetra valence, two for CD16A and two for CD30, TandAb has risks for non-specifically activating CD16A in the absence of CD30 and inducing side effects.

### SUMMARY

In one aspect, provided is a single domain antibody specifically binding to CD16A, wherein the single domain antibody has an amino acid sequence shown in SEQ ID NO: 1 comprising a substitution at a position selected from S54, G55, S56 and any combination thereof according to Kabat numbering system.

In some embodiments, the substitution is selected from a group consisting of (i) S54N, S54D or S54T, (ii) G55V, (iii) S56Q, S56T, S56D or S56E, and any combination thereof.

In some embodiments, the substitution is selected from a group consisting of: (i) S54N; (ii) S54D; (iii) S54T; (iv) G55V; (v) S56Q; (vi) S56T; (vii) S56D; (viii) S56E; (ix) S54N and S56Q; and (x) S54T and S56Q.

In one aspect, provided is a single domain antibody specifically binding to CD16A, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

In some embodiments, the single domain antibody comprises an amino acid sequence selected from a group consisting of SEQ ID NO: 3, 4, 5, 6, 7, 8, 9, 10, 39, 40, and an amino acid sequence having at least 80% identity to each thereof.

In some embodiments, the single domain antibody is a humanized antibody, a human antibody, a chimeric antibody, or a camelized antibody.

Another aspect of the disclosure provides a heavy chain antibody comprising a single domain antibody as disclosed herein and an Fc portion linked to the single domain antibody.

Another aspect of the disclosure provides a bispecific antibody comprising a single domain antibody as disclosed herein.

In some embodiments, the bispecific antibody comprises the single domain antibody binding specifically to CD16A, and a second binding domain specifically binding to a second target selected from a tumor associated antigen and a tumor specific antigen.

In some embodiments, the second target is selected from a group consisting of HER2, 5T4, PSMA, BCMA, FGFR, CD20, CD33, CD19, CD22, CD123, CD30, GPC-3, CEA, EGFR1, EGFR2, EGFR3, TGF-β, ROR1, PD-L1, Claudin18.2, EpCAM, GD2, MSLN, EGFR, MUC1, MUC2, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, CA125, MG7-Ag, NY-CO-1, RCAS 1, SDCCAG16, TAAL6 and TAG72.

In some embodiments, the single domain antibody is linked to a complete IgG antibody, optionally via a peptide linker. In some embodiments, the peptide linker consists of glycine and serine residues. In some embodiments, the single domain antibody is linked to the C terminus of a heavy chain, the C terminus of a light chain, or the N terminus of a heavy chain, of the complete IgG antibody.

In some embodiments, the complete IgG antibody is an anti-HER2 antibody or an anti-5T4 antibody. In some embodiments, the anti-HER2 antibody comprises:
(a) a heavy chain having an amino acid sequence shown in SEQ ID NO: 12, and a light chain having an amino acid sequence shown in SEQ ID NO: 13;
(b) a heavy chain having an amino acid sequence shown in SEQ ID NO: 37, and a light chain having an amino acid sequence shown in SEQ ID NO: 38;
(c) a heavy chain having an amino acid sequence shown in SEQ ID NO: 25, and a light chain having an amino acid sequence shown in SEQ ID NO: 13; or
(d) a heavy chain having an amino acid sequence shown in SEQ ID NO: 26, and a light chain having an amino acid sequence shown in SEQ ID NO: 13.

In some embodiments, the bispecific antibody comprises:
(a) a heavy chain having an amino acid sequence shown in SEQ ID NO: 14, and a light chain having an amino acid sequence shown in SEQ ID NO: 13;
(b) a heavy chain having an amino acid sequence shown in SEQ ID NO: 15, and a light chain having an amino acid sequence shown in SEQ ID NO: 13;
(c) a heavy chain having an amino acid sequence shown in SEQ ID NO: 17, and a light chain having an amino acid sequence shown in SEQ ID NO: 13;
(d) a heavy chain having an amino acid sequence shown in SEQ ID NO: 12, and a light chain having an amino acid sequence shown in SEQ ID NO: 16;
(e) a heavy chain having an amino acid sequence shown in SEQ ID NO: 18, and a light chain having an amino acid sequence shown in SEQ ID NO: 13;
(f) a heavy chain having an amino acid sequence shown in SEQ ID NO: 19, and a light chain having an amino acid sequence shown in SEQ ID NO: 13;
(g) a heavy chain having an amino acid sequence shown in SEQ ID NO: 20, and a light chain having an amino acid sequence shown in SEQ ID NO: 13;
(h) a heavy chain having an amino acid sequence shown in SEQ ID NO: 21, and a light chain having an amino acid sequence shown in SEQ ID NO: 13;
(i) a heavy chain having an amino acid sequence shown in SEQ ID NO: 22, and a light chain having an amino acid sequence shown in SEQ ID NO: 13;
(j) a heavy chain having an amino acid sequence shown in SEQ ID NO: 23, and a light chain having an amino acid sequence shown in SEQ ID NO: 13;
(k) a heavy chain having an amino acid sequence shown in SEQ ID NO: 24, and a light chain having an amino acid sequence shown in SEQ ID NO: 13;
(l) a heavy chain having an amino acid sequence shown in SEQ ID NO: 27, and a light chain having an amino acid sequence shown in SEQ ID NO: 13; or
(m) a heavy chain having an amino acid sequence shown in SEQ ID NO: 28, and a light chain having an amino acid sequence shown in SEQ ID NO: 13.

In some embodiments, the anti-5T4 antibody comprises a heavy chain having an amino acid sequence shown in SEQ ID NO: 29, and a light chain having an amino acid sequence shown in SEQ ID NO: 30.

In some embodiments, the bispecific antibody comprises a heavy chain having an amino acid sequence shown in SEQ ID NO: 31, and a light chain having an amino acid sequence shown in SEQ ID NO: 30.

Another aspect of the disclosure provides an immunoconjugate, a pharmaceutically acceptable salt thereof, or a solvate thereof, comprising a single domain antibody as disclosed herein, a heavy chain antibody as disclosed herein, or a bispecific antibody as disclosed herein; and an active moiety.

In some embodiments, the active moiety is selected from a group consisting of a toxin, a peptide tag, sortag, a radionuclide, a near-infrared fluorochromes, and a nanoparticle.

Another aspect of the disclosure provides a pharmaceutical composition comprising a single domain antibody as disclosed herein, a heavy chain antibody as disclosed herein, a bispecific antibody as disclosed herein, or an immunoconjugate as disclosed herein; and a pharmaceutically acceptable carrier.

Further aspects of the disclosure provide a nucleic acid molecule encoding a single domain antibody as disclosed herein, a heavy chain antibody as disclosed herein, or a bispecific antibody as disclosed herein, or an immunoconjugate as disclosed herein; an expression vector comprising the nucleic acid molecule as disclosed herein; and a non-human host cell comprising the expression vector as disclosed herein.

Further aspect of the disclosure provides use of a single domain antibody as disclosed herein, a heavy chain antibody as disclosed herein, a bispecific antibody as disclosed herein, an immunoconjugate as disclosed herein, or a composition as disclosed herein in the preparation of a medicament for treatment of a cancer.

Further aspect of the disclosure provides a single domain antibody as disclosed herein, a heavy chain antibody as disclosed herein, a bispecific antibody as disclosed herein, an immunoconjugate as disclosed herein, or a composition as disclosed herein for use in treatment of a cancer, acute or chronic infection, or aging-associated ailment.

Further aspect of the disclosure provides a method for treatment of cancer, acute or chronic infection, or aging-associated ailment comprising administering to a subject in need thereof a therapeutically effective amount of a single domain antibody as disclosed herein, a heavy chain antibody as disclosed herein, a bispecific antibody as disclosed herein, an immunoconjugate as disclosed herein, or a composition as disclosed herein.

Further aspect of the disclosure provides a polypeptide comprising a single domain antibody as disclosed herein, or a heavy chain antibody as disclosed herein; and one or more amino acid residues covalently linked to the N terminus, C terminus or anywhere therebetween of the single domain antibody as disclosed herein or the heavy chain antibody as disclosed herein.

These and other aspects and advantageous of the present disclosure will be apparent from the detailed description of the invention that follows.

### BRIEF DESCRIPTION OF THE FIGURES

FIG. 1: Binding of selected VHs to CD16A 158V and CD16A 158F on ELISA. BM156-01 as control.

FIG. 2: Binding affinities of selected VHs to CD16A 158V and CD16A 158F were determined by BLI.

FIG. 3. Diagram of different bispecific formats (A) A preferred anti-CD16 VH (B) Trastuzumab IgG1, (Seq ID NO: 12 and Seq ID NO: 13) (C) A homodimeric construct where CD16 VH is fused to the C terminus of heavy chain (Seq ID NO: 14 and Seq ID NO: 13). (D) A variety of G4S linkers could be used to link VH to C terminus of heavy chain (Seq ID NO: 15 and Seq ID NO: 13). (E) A homodimeric construct where CD16 VH is fused to the C terminus of light chain (Seq ID NO: 12 and Seq ID NO: 16). (F) A homodimeric construct where CD16 VH is fused to the N terminus of heavy chain with a (G4S) 3 linker (Seq ID NO: 17 and Seq ID NO: 13).

FIG. 4: binding of bispecific antibodies to human CD16A 158F (left panel) and CD16A 158V (right panel) on ELISA.

FIG. 5: Binding of different bispecific antibodies to CD16A 158V and CD16A 158F determined by BLI.

FIG. 6: Dual binding of BMP01-16 to HER2 and CD16A on ELISA.

FIG. 7: Binding of BMP01-16 to human CD16A-158V and CD16B on ELISA. BM130-92 (Margetuximab) and BM130-93 (Trastuzumab) were used as controls.

FIG. 8: Binding of different bispecific antibodies to HER2 receptor expressed on SK-BR-3, JIMT-1 and MDA-MB-231 cell surface.

FIG. 9. BMP01 exhibits higher ADCC potency than trastuzumab measured by ADCC reporter assay.

FIG. 10. LALA and N297A mutations don't abolish BMP01-12 ADCC activity in ADCC reporter assay.

FIG. 11. BMP02-10 exhibits higher ADCC potency than m603 measured by ADCC reporter assay.

FIG. 12: BMP01 bispecific antibodies enhance ADCC cell killing comparing to Trastuzumab.

FIG. 13: BMP02-10 bispecific antibodies enhance ADCC cell killing comparing to m603.

FIG. 14: BMP01 bispecific antibodies enhance IFN-γ production comparing to Trastuzumab.

FIG. 15: BMP01-16 showed stronger anti-tumor activity than BMP130-93 at low dose in JIMT-1/PBMC co-Mix tumor model. Average tumor growth for all groups was plotted on top and tumor growth of individual mouse for PBMC vs PBMC+BM130-93 and PBMC vs PBMC+BMP01-16 were plotted on bottom.

FIG. 16: BMP01-16 inhibits HCC1954 tumor growth in huHSC-NCG-hIL 15 model. Average tumor growth for all groups was plotted on top and tumor growth of individual mouse for Ctrl vs BM130-93 and Ctrl vs BMP01-16 were plotted on bottom.

### DETAILED DESCRIPTION OF THE INVENTION

### Definition

Unless stated otherwise, the following terms and phrases as used herein are intended to have the following meanings.

As used herein, the term “complete IgG antibody” generally refers to a tetramer comprising at least two heavy (H) chains and two light (L) chains inter-connected by disulfide bonds. Each heavy chain (HC) is comprised of a heavy chain variable region (abbreviated herein as VH) and a heavy chain constant region. The heavy chain constant region is comprised of three domains, CH1, CH2 and CH3. Each light chain (LC) is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “complete IgG antibody” includes, but is not limited to, monoclonal antibodies, human antibodies, humanized antibodies, and chimeric antibodies. The antibodies can be of any isotype/class (e.g., IgG, IgE, IgM, IgD, IgA and IgY) or subclass (e.g., IgG1, IgG2, IgG3, IgG4, IgA1 and IgA2).

A “heavy chain antibody” is devoid of a light chain and in the case of the heavy chain antibodies in camelids the CH1 domain is also missing. Consequently, a camelid heavy chain antibody associates with its cognate antigen via a single domain, the variable heavy chain domain of a heavy chain antibody or VHH. The terms VHH, VH, nanobody or single domain antibody are used interchangeably herein. Therefore, an anti-CD16A VH is an anti-CD16A VHH, or an anti-CD16A nanobody, or an anti-CD16A single domain antibody. Throughout the disclosure, CD16A is sometime referred to CD16. VHH antibodies are the isolated VHH domains of heavy-chain only antibodies produced by camelid species, such as alpaca, llama, and camel. These proteins consist of framework regions and three highly variable loops: CDR1, CDR2 and CDR3. Research has shown that it is the CDR3 loop that enables VHH antibodies to have such a wide range of bind specificities; the CDR3 loop tends to be 3-4 residues longer than that of conventional antibodies, allowing them to use these protrusions to extend into “hidden” epitope cavities; the CDR3 loop also has more variation in amino acid sequences in VHH antibodies than conventional antibodies, leading to ~7% higher sequence diversity per residue.

The term “Fc portion” refers to a polypeptide comprising a C-terminal part of an immunoglobulin H chain and retaining at least one functionality of a Fc-region of an IgG region, in particular the function of binding to FcRn. The antibody effector functions are determined by sequences in the Fc region. A Fc portion can comprise a CH2 domain, a CH3 domain or a CH2-CH3 polypeptide chain. The CH2-CH3 polypeptide chain assembles with another CH2-CH3 polypeptide chain to a dimer of two CH2-CH3 polypeptides combined with one another, wherein the dimerization is promoted by covalent linkage in the Hinge region NC-terminal to the CH2 domain. Hence, in some embodiments the Fc portion comprises a dimer of two CH2-CH3 polypeptide chains and a Hinge region. Preferably, the Fc portion comprises constant domains of the Ig class, e.g., IgA, IgD, IgE, IgM, preferably IgG1, IgG2, IgG3, IgG4, in particular IgG1 constant domains. An example of an Fc portion has the amino acid sequence as depicted in SEQ ID NO: 89.

The “hinge” domain may be of the same or different IgG class as the Fc portion or an engineered, not naturally occurring Hinge domain. An example of an IgG Hinge region has the amino acid sequence as depicted in SEQ ID NO:90. Included are also variants of wild-type Hinge regions, such as shortened Hinge regions.

“Linker” refers to a sequence of amino acids comprising a linker peptide joining two juxtaposed variable domains forming an antigen-binding moiety with the C-terminus of one domain linked to the N-terminus of the other juxtaposed domain or vice versa. Regarding the amino acid composition, a peptide linker sequence is selected that does not interfere with the formation of Fv, i.e. VH/VL, antigen binding- and recognition sites as well as does not interfere with the multimerization, e.g., dimerization of the polypeptides of a multispecific antigen-binding protein. For example, a linker comprising glycine and serine residues generally provides protease resistance. In some embodiments (G2S)_{X} peptide linkers are used, wherein, for example, x=1-20, e.g., (G2S), (G2S)₂, (G2S)₃, (G2S)₄, (G2S)₅, (G2S)₆, (G2S)₇ or (G2S)₈, or (G3S)_{X} peptide linkers are used, wherein, for example, x=1-15 or (G4S)_{X} peptide linkers are used, wherein, for example, x=1-10, preferably 1-6. The amino acid sequence of the linker can be optimized, for example, by phage-display methods to improve the formation of the antigen binding site and production yield of the polypeptide.

The term “target” or “target antigen” refers to an antigen which is expressed by or associated with a type of cell, i.e., target cell, or virus-infected cell to which the NK cells should be directed to induce or trigger the NK cell cytotoxicity. Examples of a target antigen may be tumor specific antigen (TSA) or tumor associated antigen (TAA). The TSA or TAA may be expressed on the surface of the target cell or displayed by an MHC complex as an MHC-restricted peptide. Examples of tumor antigens include but are not limited to HER2, 5T4, PSMA, BCMA, FGFR, CD20, CD33, CD19, CD22, CD123, CD30, GPC-3, CEA, EGFR1, EGFR2, EGFR3, TGF-β, ROR1, PD-L1, Claudin18.2, EpCAM, GD2, MSLN, EGFR, MUC1, MUC2, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, CA125, MG7-Ag, NY-CO-1, RCAS 1, SDCCAG16, TAAL6 and TAG72.

The term “binding domain” characterizes in connection with the present invention a domain which (specifically) binds to/interacts with/recognizes a given target epitope or a given target side on the target molecules (antigens), e.g., CD16 and a target cell surface antigen, respectively. The structure and function of the first binding domain (recognizing e.g., CD16), and preferably also the structure and/or function of the second binding domain (recognizing the target cell surface antigen), is/are based on the structure and/or function of an antibody, e.g., of a full-length or whole immunoglobulin molecule and/or is/are drawn from the variable heavy chain (VH) and/or variable light chain (VL) domains of an antibody or fragment thereof. Preferably the first binding domain is characterized by the presence of three light chain CDRs (i.e., CDR1, CDR2 and CDR3 of the VL region) and/or three heavy chain CDRs (i.e. CDR1, CDR2 and CDR3 of the VH region). The second binding domain preferably also comprises the minimum structural requirements of an antibody which allow for the target binding.

The term “(specifically) binds to”, “(specifically) recognizes,” “is (specifically) directed to,” or “(specifically) reacts with” means in accordance with this invention that a binding domain interacts or specifically interacts with a given epitope or a given target side on the target molecules (antigens), e.g. CD16A, and the target cell surface antigen, e.g., HER2, respectively.

The term “does not essentially/substantially bind” or “is not capable of binding” means that a binding domain of the present invention does not bind a protein or antigen other than CD16A, and/or the target cell surface antigen, i.e., does not show reactivity of more than about 30%, preferably not more than about 20%, more preferably not more than about 10%, particularly preferably not more than about 9%, about 8%, about 7%, about 6% or about 5% with proteins or antigens other than CD16A, and/or the target cell surface antigen, whereby binding to CD16A, and/or the target cell surface antigen, respectively, is set to be about 100%.

As used herein, the term “complementarity determining domains” is used herein interchangeably with the term “complementary determining regions” (“CDRs”), and generally refers to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VH CDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4^{th} ed., Chapter 4. W.H. Freeman & Co., New York, 2000). In the art, the CDR of the antibody can be defined by various methods/schemes, such as a Kabat definition scheme based on sequence variability (see Kabat et al., protein sequence in immunology, 5^{th} edition, National Institutes of Health, Bethesda, Maryland (1991)), a Chothia definition scheme based on the position of a structural loop region (see A1-Lazikani et al., Jmol Biol 273:927-48, 1997) and a IMGT definition scheme based on the concept of IMGT-ONTOLOGY and IMGT Scientific chart rules. In certain embodiments, the present application uses the IMGT rules to define the CDRs of an antibody. The definition rules of Martin, PyIgClassify and Combined definition rules of Kabat, Chothia, IMGT, Martin and PyIgClassify are also included in this application (see Mark L. Chiu et al., Antibodies 8 (4), 55, 2019).

TABLE A
Comparation of CDR numbering among different
definition schemes (in Chothia numbering)

| CDR | Kabat | Chothia | IMGT | Martin | PyIgClassify | Combined |
|---|---|---|---|---|---|---|
| LCDR1 | L24-L34 | L24-L34 | L27-L32 | L24-L34 | L24-L34 | L24-L34 |
| LCDR2 | L50-L56 | L50-L56 | L50-L52 | L50-L56 | L49-L56 | L49-L56 |
| LCDR3 | L89-L97 | L89-L97 | L89-L97 | L89-L97 | L89-L97 | L89-L97 |
| HCDR1 | H31-H35 | H26-H32 | H26-H33 | H26-H35 | H23-H35 | H23-H35 |
| HCDR2 | H50-H65 | H52-H56 | H51-H57 | H50-H58 | H50-H58 | H50-H65 |
| HCDR3 | H95-H102 | H95-H102 | H93-H102 | H95-H102 | H93-H102 | H93-H102 |

wherein, Laa-Lbb or Haa-Hbb may refer to, from N-terminal, the amino acids sequence from NO.aa to NO.bb of light chain or heavy chain respectively. For example, L24-L34 refers to the amino acid sequence from NO. 24 to NO. 34 of light chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

As used herein, the term “humanized antibody” generally refers to an antibody which includes sequences of heavy chain variable regions and light chain variable regions derived from non-human species (e.g., mice), but in which at least a portion of the VH and/or VL sequences have been changed to be similar to the human germline variable sequences. For example, the term “humanized antibody” is an antibody or a variant, derivative, analogue or fragment thereof that can bind to a related antigen with immune specificity and includes a framework region (FR) which includes substantially an amino acid sequence of a human antibody and a complementary determining region (CDR) which includes substantially an amino acid sequence of a non-human antibody. In the context of CDR, the term “substantially” means that the amino acid sequence of CDR has at least 80%, e.g., at least 85%, at least 90%, at least 95%, at least 98% or at least 99% identity with an amino acid sequence of CDR of a non-human antibody. The humanized antibodies include substantially at least one, typically two variable domains (Fab, Fab′, F(ab′)₂, Fab, Fv), wherein all or substantially all CDR regions correspond to the CDR region of a non-human immunoglobulin and all or substantially all framework regions are frame regions with consensus sequence of human immunoglobulin. In some embodiments, the humanized antibody can further include at least a portion of a constant region of immunoglobulin (Fc), typically a constant region of human immunoglobulin.

As used herein, the term “human antibody” generally refers to an antibody with variable and constant regions derived from the sequence of immunoglobulin of human germ line. Human antibodies are well known in the prior art (e.g., van Dijk, M. A. and van de Winkel, J. G., Curr.Opin. Chem.Biol.5 (2001) 368-374). The human antibodies can also be generated in transgenic animals (e.g., mice) which can generate a complete or selected set of human antibodies in absence of generated endogenous immunoglobulin after immunization (e.g., Jakobovits, A. et al., Proc. Natl. Acad. Sci. USA 90 (1993) 2551-2555; Jakobovits, A. et al., Nature 362 (1993) 255-258; Brueggemann, M. et al., YearImmunol. 7 (1993) 33-40). The human antibodies can also be generated in a phage display library (e.g., Hoogenboom, H. R. and Winter, G., J.Mol.Biol. 227 (1992) 381-388; Marks, J. D. et al., J.Mol.Biol. 222 (1991) 581-597). The term “human antibody” can also include antibodies modified in the constant regions.

As used herein, the term “chimeric antibody” generally refers to an engineered antibody which in its broadest sense contains one or more regions from one antibody and one or more regions from one or more other antibody(ies). In particular a chimeric antibody comprises a VH domain and a VL domain of an antibody derived from a non-human animal, in association with a CH domain and a CL domain of another antibody, in particular a human antibody. As the non-human animal, any animal such as camel, mouse, rat, hamster, rabbit or the like can be used.

The term “bispecific antibody” as used herein refers to an antibody having two specificities to one or more antigens or to different epitopes of the same antigen. For example, a bispecific antibody may comprise a first specificity to a first antigen (e.g., CD16A) and a second specificity to a second antigen (e.g., a tumor associated antigen or tumor specific antigen) different from the first antigen. For example, a bispecific antibody may comprise a first specificity to a first epitope of an antigen and a second specificity to a second different epitope of the same antigen (e.g., CD16A).

By “purified” and “isolated” it is meant, when referring to a polypeptide or a nucleotide sequence, that the indicated molecule is present in the substantial absence of other biological macromolecules of the same type. The term “purified” as used herein in particular means at least 75%, 85%, 95% or 98% by weight, of biological macromolecules of the same type are present. An “isolated” nucleic acid molecule that encodes a particular polypeptide refers to a nucleic acid molecule that is substantially free of other nucleic acid molecules that do not encode the subject polypeptide; however, the 14ytoxan14e may include some additional bases or moieties, which do not deleteriously affect the basic characteristics of the composition.

The present invention may contain, for example, an isolated antigen-binding protein, an isolated antibody or an antigen-binding fragment thereof, an isolated polypeptide, an isolated nucleic acid molecule or molecules.

As used herein, the term “affinity” is generally defined by the equilibrium association between the whole antibody and the antigen. Affinity may be expressed for example in half-maximal effective concentration (EC₅₀) or the equilibrium dissociation constant (K_{D}). Affinity can be experimentally assessed by a variety of known methods, such as measuring association and dissociation rates with surface Plasmon resonance or measuring the EC₅₀ in an immunochemical assay (ELISA, FACS).

The term “conservatively modified variant” applies to both amino acid and nucleic acid sequences. With respect to particular nucleic acid sequences, conservatively modified variant refers to those nucleic acids which encode identical or essentially identical amino acid sequences or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

As used herein, the term “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95% or 99% identity over a specified region or, when not specified, over the entire sequence), when compared and aligned for maximum correspondence over a comparison window or designated region as measured using one of the following sequence comparison algorithms or by manual alignment and visual inspection. Optionally, the identity exists over a region that is at least about 30 nucleotides (or 10 amino acids) in length or more preferably over a region that is 100 to 500 or 1000 or more nucleotides (or 20, 50, 200 or more amino acids) in length. Two examples of algorithms that are suitable for determining percent sequence identity and sequence similarity are the BLAST and BLAST 2.0 algorithms, which are described in Altschul et al, Nuc. Acids Res. 25:3389-3402, 1977; and Altschul et al, J. Mol. Biol. 215:403-410, 1990, respectively.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used herein, the term “nucleic acid molecule” is used herein interchangeably with the term “polynucleotide” and generally refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, the term “polypeptide” is used herein interchangeably with the term “protein” and refers to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

As used herein, the term “immunoconjugate” as used herein generally refers to the linkage of an antibody or an antigen binding fragment thereof with another agent, such as a payload, a drug moiety, a chemotherapeutic agent, a toxin, an immunotherapeutic agent, an imaging probe, and the like. The linkage can be a covalent bond or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to a protein created through the joining of two or more genes or gene fragments which originally coded for separate proteins (including peptides and polypeptides). Translation of the fusion gene results in a single protein with functional properties derived from each of the original proteins.

As used herein, the term “toxin,” “cytotoxin” or “cytotoxic agent” as used herein, generally refers to any agent that is detrimental to the growth and proliferation of cells and may act to reduce, inhibit or destroy a cell or malignancy.

As used herein, “cancer” or “tumor” as used interchangeably herein is meant to a group of diseases which can be treated according to the disclosure and involve abnormal cell growth with the potential to invade or spread to other parts of the body. Not all tumors are cancerous; benign tumors do not spread to other parts of the body. Possible signs and symptoms include: a new lump, abnormal bleeding, a prolonged cough, unexplained weight loss, and a change in bowel movements among others. There are over 100 different known cancers that affect humans. As used herein, “cancer” includes, without limitation, a solid cancer (e.g., a tumor) and a hematologic malignancy. A “hematologic malignancy”, also known as a blood cancer, is a cancer that originates in blood-forming tissue, such as the bone marrow or other cells of the immune system. Hematologic malignancies include, without limitation, leukemias (such as acute myeloid leukemia (ANIL), acute promyelocytic leukemia, acute lymphoblastic leukemia (ALL), acute mixed lineage leukemia, chronic myeloid leukemia, chronic lymphocytic leukemia (CLL), hairy, cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies. “Solid cancers” include, without limitation, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma.

As used herein, the term “anti-tumor agent” or “antitumor drug” as used herein generally refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

As used herein, the term “anti-tumor activity” means a reduction in the rate of tumor cell proliferation, viability or metastatic activity. A possible way of showing anti-tumor activity is to show a decline in growth rate of tumor cells, tumor size stasis or tumor size reduction. Such activity can be assessed using accepted in vitro or in vivo tumor models, including but not limited to xenograft models, allograft models, MMTV models, and other known models known in the art to investigate anti-tumor activity.

As used herein, the term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

As used herein, the term “pharmaceutically acceptable” generally refers to one or more non-toxic substances that do not interfere with the effectiveness of the biological activity of the active ingredient. Such formulations may typically contain salts, buffers, preservatives, compatible carriers, and optionally other therapeutic agents. Such pharmaceutically acceptable formulations may also typically contain compatible solid or liquid fillers, diluents, or encapsulating materials suitable for administration to humans. When used in medicine, the salt should be a pharmaceutically acceptable salt, but non-pharmaceutically acceptable salts can be conveniently used to prepare pharmaceutically acceptable salts and cannot be excluded from the scope of the present invention. Such pharmacologically and pharmaceutically acceptable salts include, but are not limited to, salts prepared from the following acids: hydrochloric acid, hydrobromic acid, sulfuric acid, nitric acid, phosphoric acid, maleic acid, acetic acid, salicylic acid, citric acid, boric acid, formic acid, malonic acid, succinic acid, etc. Pharmacologically acceptable salts can also be prepared as alkali metal salts or alkaline earth metal salts, such as sodium, potassium or calcium salts. The term “solvate” is used herein in the conventional sense to refer to a complex of a solute (e.g., an active compound, a salt of an active compound) and a solvent. Solvates usually do not significantly alter the physiological activity or toxicity of the compound and therefore can act as pharmacological equivalents. If the solvent is water, the solvent compound may be conveniently referred to as a hydrate, e.g., monohydrate, dihydrate, trihydrate, etc.

As used herein, the terms “treat,” “treating,” or “treatment” of any disease or disorder generally refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter) or both. In yet another aspect, “treat,” “treating,” or “treatment” refers to preventing or delaying the onset or development or progression of the disease or disorder.

As used herein, the term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some embodiments, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of or result in a decrease in severity of, respectively, disease symptoms, including symptoms associated with cancer.

As used herein, the terms “comprising”, “containing”, “having”, “include”, and “including” are to be construed as “including, but not limited to” unless otherwise noted. The terms “a,” “an,” and “the” and similar referents in the context of describing the invention and, specifically, in the context of the appended claims, are to be construed to cover both the singular and the plural unless otherwise noted. The use of any and all examples or exemplary language (“for example”, “e.g.”, “such as”) is intended merely to illustrate aspects or embodiments of the invention, and is not to be construed as limiting the scope thereof, unless otherwise claimed.

As used herein, the term “about” when referring to a measurable value such as an amount, a temporal duration, and the like, is meant to encompass variations of ±20% or in some instances±10% or in some instances±5% or in some instances±1% or in some instances±0.1% from the specified value, as such variations are appropriate to perform the disclosed methods.

The term “vector” includes shuttle and expression vectors. Typically, a vector could be a plasmid construct that also include an origin of replication (e.g., the Col E1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the present disclosure, in bacterial or eukaryotic cells.

### Anti-CD16A Single Domain Antibodies

In one aspect, the present disclosure provides an anti-CD16A single domain antibody, wherein the single domain antibody has an amino acid sequence shown in SEQ ID NO: 1 comprising a substitution at a position selected from S54, G55, S56 and any combination thereof according to Kabat numbering system.

In some embodiments, the anti-CD16A single domain antibody has an amino acid sequence shown in SEQ ID NO: 1 comprising a substitution at a position S54. In some embodiments, the anti-CD16A single domain antibody has an amino acid sequence shown in SEQ ID NO: 1 comprising a substitution at a position G55. In some embodiments, the anti-CD16A single domain antibody has an amino acid sequence shown in SEQ ID NO: 1 comprising a substitution at a position S56.

In some embodiments, the anti-CD16A single domain antibody has an amino acid sequence shown in SEQ ID NO: 1 comprising substitutions at positions S54 and G55. In some embodiments, the anti-CD16A single domain antibody has an amino acid sequence shown in SEQ ID NO: 1 comprising substitutions at positions S54 and S56. In some embodiments, the anti-CD16A single domain antibody has an amino acid sequence shown in SEQ ID NO: 1 comprising substitutions at positions G55 and S56. In some embodiments, the anti-CD16A single domain antibody has an amino acid sequence shown in SEQ ID NO: 1 comprising substitutions at positions S54, G55 and S56.

In some embodiments, the anti-CD16A single domain antibody has an amino acid sequence shown in SEQ ID NO: 1 comprising a substitution at positions S54, G55 and S56, and one or more further substitution at positions other than S54, G55, and S56. In these embodiments, the one or more further substitution is preferably a conservative substitution.

In some embodiments, the serine residue at position 54 is substituted by an amino acid residue other than serine. For example, the serine residue at position 54 is substituted by any of alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), threonine (T), tryptophan (W), tyrosine (Y) or valine (V). Preferably, the serine residue at position 54 is substituted by asparagine (N), aspartic acid (D), or threonine (T).

In some embodiments, the glycine residue at position 55 is substituted by an amino acid residue other than glycine. For example, the glycine residue at position 55 is substituted by any of alanine (A), arginine (R), asparagine (N), aspartic acid (D), cysteine (C), glutamine (Q), glutamic acid (E), histidine (H), isoleucine (I), leucine (L), lysine (K), methionine (M), phenylalanine (F), proline (P), serine(S), threonine (T), tryptophan (W), tyrosine (Y) or valine (V). Preferably, the glycine residue at position 55 is substituted by valine (V).

In some embodiments, the serine residue at position 56 is substituted by an amino acid residue other than serine. For example, the serine residue at position 56 is substituted by any of alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), threonine (T), tryptophan (W), tyrosine (Y) or valine (V). Preferably, the serine residue at position 56 is substituted by glutamine (Q), threonine (T), aspartic acid (D), or glutamic acid (E).

In some embodiments, the substitution is selected from a group consisting of (i) S54N, S54D or S54T; (ii) G55V; (iii) S56Q, S56T, S56D or S56E; and any combination thereof.

In some embodiments, the single domain antibody has an amino acid sequence shown in SEQ ID NO: 1 with a single substitution of S54N according to Kabat numbering system. In some embodiments, the single domain antibody has an amino acid sequence shown in SEQ ID NO: 1 with a single substitution of S54D according to Kabat numbering system. In some embodiments, the single domain antibody has an amino acid sequence shown in SEQ ID NO: 1 with a single substitution of S54T according to Kabat numbering system.

In some embodiments, the single domain antibody has an amino acid sequence shown in SEQ ID NO: 1 with a single substitution of G55V according to Kabat numbering system.

In some embodiments, the single domain antibody has an amino acid sequence shown in SEQ ID NO: 1 with a single substitution of S56Q according to Kabat numbering system. In some embodiments, the single domain antibody has an amino acid sequence shown in SEQ ID NO: 1 with a single substitution of S56T according to Kabat numbering system. In some embodiments, the single domain antibody has an amino acid sequence shown in SEQ ID NO: 1 with a single substitution of S56D according to Kabat numbering system. In some embodiments, the single domain antibody has an amino acid sequence shown in SEQ ID NO: 1 with a single substitution of S56E according to Kabat numbering system.

In some embodiments, the substitution is selected from a group consisting of: (i) S54N; (ii) S54D; (iii) S54T; (iv) G55V; (v) S56Q; (vi) S56T; (vii) S56D; (viii) S56E; (ix) S54N and S56Q; and (x) S54T and S56Q.

In some embodiments, the single domain antibody has an amino acid sequence shown in SEQ ID NO: 1 with two substitutions of S54N and S56Q according to Kabat numbering system. In some embodiments, the single domain antibody has an amino acid sequence shown in SEQ ID NO: 1 with two substitutions of S54T and S56Q according to Kabat numbering system.

In some embodiments, the single domain antibody harboring a substitution at a position selected from S54, G55, S56 and any combination thereof comprises an amino acid sequence selected from a group consisting of SEQ ID NO: 3, 4, 5, 6, 7, 8, 9, 10, 39, 40, and an amino acid sequence having at least 80%, at least 85%, at least 90%, at least 95%, at least 96%, at least 97%, at least 98%, or at least 99% identity to each thereof.

In some embodiments, the single domain antibody harboring a substitution at a position selected from S54, G55, S56 and any combination thereof comprises an amino acid sequence shown in SEQ ID NO: 3, or an amino acid sequence having at least 80%, at least 85%, at least 90%, at least 95%, at least 96%, at least 97%, at least 98%, or at least 99% identity to SEQ ID NO: 3.

In some embodiments, the single domain antibody harboring a substitution at a position selected from S54, G55, S56 and any combination thereof comprises an amino acid sequence shown in SEQ ID NO: 4, or an amino acid sequence having at least 80%, at least 85%, at least 90%, at least 95%, at least 96%, at least 97%, at least 98%, or at least 99% identity to SEQ ID NO: 4.

In some embodiments, the single domain antibody harboring a substitution at a position selected from S54, G55, S56 and any combination thereof comprises an amino acid sequence shown in SEQ ID NO: 5, or an amino acid sequence having at least 80%, at least 85%, at least 90%, at least 95%, at least 96%, at least 97%, at least 98%, or at least 99% identity to SEQ ID NO: 5.

In some embodiments, the single domain antibody harboring a substitution at a position selected from S54, G55, S56 and any combination thereof comprises an amino acid sequence shown in SEQ ID NO: 6, or an amino acid sequence having at least 80%, at least 85%, at least 90%, at least 95%, at least 96%, at least 97%, at least 98%, or at least 99% identity to SEQ ID NO: 6.

In some embodiments, the single domain antibody harboring a substitution at a position selected from S54, G55, S56 and any combination thereof comprises an amino acid sequence shown in SEQ ID NO: 7, or an amino acid sequence having at least 80%, at least 85%, at least 90%, at least 95%, at least 96%, at least 97%, at least 98%, or at least 99% identity to SEQ ID NO: 7.

In some embodiments, the single domain antibody harboring a substitution at a position selected from S54, G55, S56 and any combination thereof comprises an amino acid sequence shown in SEQ ID NO: 8, or an amino acid sequence having at least 80%, at least 85%, at least 90%, at least 95%, at least 96%, at least 97%, at least 98%, or at least 99% identity to SEQ ID NO: 8.

In some embodiments, the single domain antibody harboring a substitution at a position selected from S54, G55, S56 and any combination thereof comprises an amino acid sequence shown in SEQ ID NO: 9, or an amino acid sequence having at least 80%, at least 85%, at least 90%, at least 95%, at least 96%, at least 97%, at least 98%, or at least 99% identity to SEQ ID NO: 9.

In some embodiments, the single domain antibody harboring a substitution at a position selected from S54, G55, S56 and any combination thereof comprises an amino acid sequence shown in SEQ ID NO: 10, or an amino acid sequence having at least 80%, at least 85%, at least 90%, at least 95%, at least 96%, at least 97%, at least 98%, or at least 99% identity to SEQ ID NO: 10.

In some embodiments, the single domain antibody harboring a substitution at a position selected from S54, G55, S56 and any combination thereof comprises an amino acid sequence shown in SEQ ID NO: 39, or an amino acid sequence having at least 80%, at least 85%, at least 90%, at least 95%, at least 96%, at least 97%, at least 98%, or at least 99% identity to SEQ ID NO: 39.

In some embodiments, the single domain antibody harboring a substitution at a position selected from S54, G55, S56 and any combination thereof comprises an amino acid sequence shown in SEQ ID NO: 40, or an amino acid sequence having at least 80%, at least 85%, at least 90%, at least 95%, at least 96%, at least 97%, at least 98%, or at least 99% identity to SEQ ID NO: 40.

Another aspect of the disclosure provides a single domain antibody specifically binding to CD16A, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

In some embodiments, provided is a single domain antibody specifically binding to CD16A, wherein the single domain antibody comprises CDR1 having an amino acid sequence as shown in SEQ ID NO: 41, CDR3 having an amino acid sequence as shown in SEQ ID NO: 42, and CDR2 having an amino acid sequence selected from a group consisting of SEQ ID NO: 43, 44, 45, 46, 47, 48, 49, 50, 51, and 52, according to IMGT definition scheme.

In some embodiments, provided is a single domain antibody specifically binding to CD16A, wherein the single domain antibody comprises CDR1 having an amino acid sequence as shown in SEQ ID NO: 53, CDR3 having an amino acid sequence as shown in SEQ ID NO: 54, and CDR2 having an amino acid sequence selected from a group consisting of SEQ ID NO: 55, 56, 57, 58, 59, 60, 61, 62, 63, and 64, according to Kabat definition scheme.

In some embodiments, provided is a single domain antibody specifically binding to CD16A, wherein the single domain antibody comprises CDR1 having an amino acid sequence as shown in SEQ ID NO: 65, CDR3 having an amino acid sequence as shown in SEQ ID NO: 66, and CDR2 having an amino acid sequence selected from a group consisting of SEQ ID NO: 67, 68, 69, 70, 71, 72, 73, 74, 75, and 76, according to Chothia definition scheme.

In some embodiments, provided is a single domain antibody specifically binding to CD16A, wherein the single domain antibody comprises CDR1 having an amino acid sequence as shown in SEQ ID NO: 77, CDR3 having an amino acid sequence as shown in SEQ ID NO: 78, and CDR2 having an amino acid sequence selected from a group consisting of SEQ ID NO: 79, 80, 81, 82, 83, 84, 85, 86, 87, and 88, according to Contact definition scheme.

In some embodiments, provided is a single domain antibody comprising an amino acid sequence selected from a group consisting of SEQ ID NO: 3, 4, 5, 6, 7, 8, 9, 10, 39, and 40.

In some embodiments, provided is a single domain antibody comprising an amino acid sequence having at least 80%, at least 85%, at least 90%, at least 95%, at least 96%, at least 97%, at least 98%, or at least 99% identity to SEQ ID NO: 3, 4, 5, 6, 7, 8, 9, 10, 39, or 40, and comprising CDR1 having an amino acid sequence as shown in SEQ ID NO: 41, CDR3 having an amino acid sequence as shown in SEQ ID NO: 42, and CDR2 having an amino acid sequence selected from a group consisting of SEQ ID NO: 43, 44, 45, 46, 47, 48, 49, 50, 51, and 52, according to IMGT definition scheme.

In some embodiments, provided is a single domain antibody comprising an amino acid sequence having at least 80%, at least 85%, at least 90%, at least 95%, at least 96%, at least 97%, at least 98%, or at least 99% identity to SEQ ID NO: 3, 4, 5, 6, 7, 8, 9, 10, 39, or 40, and comprising CDR1 having an amino acid sequence as shown in SEQ ID NO: 53, CDR3 having an amino acid sequence as shown in SEQ ID NO: 54, and CDR2 having an amino acid sequence selected from a group consisting of SEQ ID NO: 55, 56, 57, 58, 59, 60, 61, 62, 63, and 64, according to Kabat definition scheme.

In some embodiments, provided is a single domain antibody comprising an amino acid sequence having at least 80%, at least 85%, at least 90%, at least 95%, at least 96%, at least 97%, at least 98%, or at least 99% identity to SEQ ID NO: 3, 4, 5, 6, 7, 8, 9, 10, 39, or 40, and comprising CDR1 having an amino acid sequence as shown in SEQ ID NO: 65, CDR3 having an amino acid sequence as shown in SEQ ID NO: 66, and CDR2 having an amino acid sequence selected from a group consisting of SEQ ID NO: 67, 68, 69, 70, 71, 72, 73, 74, 75, and 76, according to Chothia definition scheme.

In some embodiments, provided is a single domain antibody comprising an amino acid sequence having at least 80%, at least 85%, at least 90%, at least 95%, at least 96%, at least 97%, at least 98%, or at least 99% identity to SEQ ID NO: 3, 4, 5, 6, 7, 8, 9, 10, 39, or 40, and comprising CDR1 having an amino acid sequence as shown in SEQ ID NO: 77, CDR3 having an amino acid sequence as shown in SEQ ID NO: 78, and CDR2 having an amino acid sequence selected from a group consisting of SEQ ID NO: 79, 80, 81, 82, 83, 84, 85, 86, 87, and 88, according to Contact definition scheme.

In any of the embodiments of anti-CD16A single domain antibody disclosed herein, the single domain antibody may be a humanized antibody, a human antibody, a chimeric antibody, or a camelized antibody. Preferably, the single domain antibody is a humanized antibody or a human antibody. More preferably, the single domain antibody is a humanized antibody.

In some embodiments, the anti-CD16A single domain antibody disclosed herein binds specifically to CD16A, but not specifically to CD16B. In some embodiments, the anti-CD16A single domain antibody disclosed herein binds specifically to human and monkey CD16A, but not specifically to mouse or rat CD16A. The amino acid sequence of human CD16A is illustratively shown in SEQ ID NO: 2 or 11. The amino acid sequence of human CD16B is illustratively shown in SEQ ID NO: 36. The amino acid sequence of monkey CD16A is illustratively shown in SEQ ID NO: 33. The amino acid sequence of mouse CD16A is illustratively shown in SEQ ID NO: 34. The amino acid sequence of rat CD16A is illustratively shown in SEQ ID NO: 35.

In some embodiments, the anti-CD16A single domain antibody disclosed herein binds specifically to CD16A 158V and 158F. In some embodiments, the anti-CD16A single domain antibody disclosed herein binds specifically to CD16A 158V and 158F at substantially same affinity. In some embodiments, the anti-CD16A single domain antibody disclosed herein binds specifically to CD16A 158V and 158F at substantially same affinity and with EC₅₀ in a range of about 1 to about 7 nM, about 1 to about 4 nM, about 1 to about 2 nM, or about 1 to about 1.5 nM, as determined by ELISA. In some embodiments, the anti-CD16A single domain antibody disclosed herein binds specifically to CD16A 158V with EC₅₀ in a range of about 1 to about 4 nM, about 1 to about 3 nM, about 1 to about 2 nM, or about 1 to about 1.5 nM, as determined by ELISA. In some embodiments, the anti-CD16A single domain antibody disclosed herein binds specifically to CD16A 158F with EC₅₀ in a range of about 1 to about 3 nM, about 1 to about 2 nM, or about 1 to about 1.2 nM, as determined by ELISA.

In some embodiments, the anti-CD16A single domain antibody disclosed herein has improved binding affinity to CD16A, especially CD16A 158V, than the anti-CD16A single domain antibody having an amino acid sequence shown in SEQ ID NO: 1. In some embodiments, the anti-CD16A single domain antibody disclosed herein binds specifically to CD16A 158V at EC₅₀ in a range of about 2 to about 16 nM, about 2 to about 10 nM, about 2 to about 8 nM, about 2 to about 5 nM, or about 2 to about 3 nM, as determined by BLI. In some embodiments, the anti-CD16A single domain antibody disclosed herein binds specifically to CD16A 158F at EC₅₀ in a range of about 1 to about 6 nM, about 1 to about 5 nM, about 1 to about 4 nM, about 1 to about 3 nM, or about 1 to about 2 nM, as determined by BLI.

In some embodiments, provided is a polypeptide comprising an anti-CD16A single domain antibody as described herein and one or more amino acid residues covalently linked to the N terminus, C terminus or anywhere therebetween of the anti-CD16A single domain antibody, and preferably the anti-CD16A single domain antibody comprises an amino acid sequence selected from a group consisting of SEQ ID NO: 3, 4, 5, 6, 7, 8, 9, 10, 39, and 40.

### Anti-CD16A Heavy Chain Antibodies

Another aspect of the disclosure provides a heavy chain antibody comprising a single domain antibody as disclosed herein, and an Fc portion linked to the single domain antibody.

In some embodiments, an anti-CD16A heavy chain antibody is provided wherein the heavy chain antibody comprises a single domain antibody comprising:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that heavy chain antibody does not comprise an amino acid sequence shown in SEQ ID NO: 1.

In some embodiments, an anti-CD16A heavy chain antibody is provided wherein the heavy chain antibody comprises an amino acid sequence selected from a group consisting of SEQ ID NO: 3, 4, 5, 6, 7, 8, 9, 10, 39, and 40.

In some embodiments, an anti-CD16A heavy chain antibody is provided wherein heavy chain antibody comprises an amino acid sequence having at least 80%, at least 85%, at least 90%, at least 95%, at least 96%, at least 97%, at least 98%, or at least 99% identity to SEQ ID NO: 3, 4, 5, 6, 7, 8, 9, 10, 39, or 40, and comprising CDR1 having an amino acid sequence as shown in SEQ ID NO: 41, CDR3 having an amino acid sequence as shown in SEQ ID NO: 42, and CDR2 having an amino acid sequence selected from a group consisting of SEQ ID NO: 43, 44, 45, 46, 47, 48, 49, 50, 51, and 52, according to IMGT definition scheme.

In some embodiments, an anti-CD16A heavy chain antibody is provided wherein the heavy chain antibody comprises a single domain antibody comprising an amino acid sequence selected from a group consisting of SEQ ID NO: 3, 4, 5, 6, 7, 8, 9, 10, 39, and 40, and the single domain antibody is linked to a Fc portion having an amino acid sequence shown in SEQ ID NO: 89.

In any of the embodiments of anti-CD16A heavy chain antibody disclosed herein, the anti-CD16A heavy chain antibody may be a humanized antibody, a human antibody, a chimeric antibody, or a camelized antibody. Preferably, the anti-CD16A heavy chain antibody is a humanized antibody or a human antibody. More preferably, the anti-CD16A heavy chain antibody is a humanized antibody.

In some embodiments, provided is a polypeptide comprising an anti-CD16A heavy chain antibody as described herein and one or more amino acid residues covalently linked to the N terminus, C terminus or anywhere therebetween of the anti-CD16A heavy chain antibody, and preferably the anti-CD16A heavy chain antibody comprises an amino acid sequence selected from a group consisting of SEQ ID NO: 3, 4, 5, 6, 7, 8, 9, 10, 39, and 40, and a Fc portion having an amino acid sequence shown in SEQ ID NO: 89.

### Bispecific Antibodies

Another aspect of the invention provides a bispecific antibody comprising a single domain antibody as disclosed herein.

In some embodiments, the bispecific antibody comprises the single domain antibody binding specifically to CD16A as disclosed herein, and a second binding domain specifically binding to a second target selected from a tumor associated antigen and a tumor specific antigen.

In some embodiments, the second target is selected from a group consisting of HER2, 5T4, PSMA, BCMA, FGFR, CD20, CD33, CD19, CD22, CD123, CD30, GPC-3, CEA, EGFR1, EGFR2, EGFR3, TGF-β, ROR1, PD-L1, Claudin18.2, EpCAM, GD2, MSLN, EGFR, MUC1, MUC2, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, CA125, MG7-Ag, NY-CO-1, RCAS 1, SDCCAG16, TAAL6 and TAG72. Preferably, the second target is HER2, 5T4, BCMA, CD20, CD19, CD30, Claudin18.2, MUC1, Trop-2, c-MET, or Nectin-4. More preferably, the second target is HER2, 5T4, or CD30.

In some embodiments, the bispecific antibody comprises a first binding domain specifically binding to CD16A, and second binding domain specifically binding to HER2, wherein the first binding domain is a single domain antibody specifically binding to CD16A, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

In some embodiments, the bispecific antibody comprises a first binding domain specifically binding to CD16A, and second binding domain specifically binding to 5T4, wherein the first binding domain is a single domain antibody specifically binding to CD16A, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

In some embodiments, the bispecific antibody comprises a first binding domain specifically binding to CD16A, and second binding domain specifically binding to CD30, wherein the first binding domain is a single domain antibody specifically binding to CD16A, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

In some embodiments, the bispecific antibody comprises a first binding domain specifically binding to CD16A, and second binding domain specifically binding to HER2, wherein the first binding domain is a single domain antibody specifically binding to CD16A, wherein the single domain antibody comprises an amino acid sequence selected from a group consisting of SEQ ID NO: 3, 4, 5, 6, 7, 8, 9, 10, 39, and 40.

In some embodiments, the bispecific antibody comprises a first binding domain specifically binding to CD16A, and second binding domain specifically binding to 5T4, wherein the first binding domain is a single domain antibody specifically binding to CD16A, wherein the single domain antibody comprises an amino acid sequence selected from a group consisting of SEQ ID NO: 3, 4, 5, 6, 7, 8, 9, 10, 39, and 40.

In some embodiments, the bispecific antibody comprises a first binding domain specifically binding to CD16A, and second binding domain specifically binding to CD30, wherein the first binding domain is a single domain antibody specifically binding to CD16A, wherein the single domain antibody comprises an amino acid sequence selected from a group consisting of SEQ ID NO: 3, 4, 5, 6, 7, 8, 9, 10, 39, and 40.

In some embodiments, the bispecific antibody comprises a first binding domain specifically binding to CD16A, and second binding domain specifically binding to a second target selected from a tumor associated antigen and a tumor specific antigen, the first binding domain is a single domain antibody specifically binding to CD16A, and the second binding domain is a complete IgG antibody or an antigen binding fragment thereof such as Fab, Fv, scFv, or F(ab′)2.

In some embodiments, the bispecific antibody comprises a first binding domain specifically binding to CD16A, and second binding domain specifically binding to a second target selected from a tumor associated antigen and a tumor specific antigen, the first binding domain is a single domain antibody specifically binding to CD16A, the second binding domain is a complete IgG antibody, and the single domain antibody is linked to the C terminus of a heavy chain, the C terminus of a light chain, or the N terminus of a heavy chain, of the complete IgG antibody.

In some embodiments, the bispecific antibody comprises a first binding domain specifically binding to CD16A, and second binding domain specifically binding to a second target selected from a tumor associated antigen and a tumor specific antigen, the first binding domain is a single domain antibody specifically binding to CD16A, the second binding domain is a complete IgG antibody, and the single domain antibody is linked to the C terminus of a heavy chain, the C terminus of a light chain, or the N terminus of a heavy chain, of the complete IgG antibody via a peptide linker.

In some embodiments, the bispecific antibody comprises a first binding domain specifically binding to CD16A, and second binding domain specifically binding to a second target selected from a tumor associated antigen and a tumor specific antigen, the first binding domain is a single domain antibody specifically binding to CD16A, the second binding domain is a complete IgG antibody, and the single domain antibody is linked to the C terminus of a heavy chain, the C terminus of a light chain, or the N terminus of a heavy chain, of the complete IgG antibody via a peptide linker consisting of glycine and serine residues. The peptide linker is as defined in the Definition section.

In some embodiments, the bispecific antibody comprises a single domain antibody specifically binding to CD16A as disclosed herein, and a complete anti-HER2 IgG antibody, and the single domain antibody is linked to the C terminus of a heavy chain, the C terminus of a light chain, or the N terminus of a heavy chain, of the complete IgG antibody via a peptide linker consisting of glycine and serine residues.

In some embodiments, the bispecific antibody comprises a single domain antibody specifically binding to CD16A as disclosed herein, and a complete anti-5T4 IgG antibody, and the single domain antibody is linked to the C terminus of a heavy chain, the C terminus of a light chain, or the N terminus of a heavy chain, of the complete IgG antibody via a peptide linker consisting of glycine and serine residues.

In some embodiment, the complete IgG antibody has a mutation in the Fc portion. In some embodiment, the complete IgG antibody has L234A L235A mutations (“LALA”). In some embodiment, the complete IgG antibody has a N297A mutation. In some embodiment, the complete IgG antibody has “LALA” and N297A mutations.

In some embodiments, the complete anti-HER2 IgG antibody comprises:
(a) a heavy chain having an amino acid sequence shown in SEQ ID NO: 12, and a light chain having an amino acid sequence shown in SEQ ID NO: 13;
(b) a heavy chain having an amino acid sequence shown in SEQ ID NO: 37, and a light chain having an amino acid sequence shown in SEQ ID NO: 38;
(c) a heavy chain having an amino acid sequence shown in SEQ ID NO: 25, and a light chain having an amino acid sequence shown in SEQ ID NO: 13; or
(d) a heavy chain having an amino acid sequence shown in SEQ ID NO: 26, and a light chain having an amino acid sequence shown in SEQ ID NO: 13.

In some embodiments, the bispecific antibody comprises:
(a) a heavy chain having an amino acid sequence shown in SEQ ID NO: 14, and a light chain having an amino acid sequence shown in SEQ ID NO: 13;
(b) a heavy chain having an amino acid sequence shown in SEQ ID NO: 15, and a light chain having an amino acid sequence shown in SEQ ID NO: 13;
(c) a heavy chain having an amino acid sequence shown in SEQ ID NO: 17, and a light chain having an amino acid sequence shown in SEQ ID NO: 13;
(d) a heavy chain having an amino acid sequence shown in SEQ ID NO: 12, and a light chain having an amino acid sequence shown in SEQ ID NO: 16;
(c) a heavy chain having an amino acid sequence shown in SEQ ID NO: 18, and a light chain having an amino acid sequence shown in SEQ ID NO: 13;
(f) a heavy chain having an amino acid sequence shown in SEQ ID NO: 19, and a light chain having an amino acid sequence shown in SEQ ID NO: 13;
(g) a heavy chain having an amino acid sequence shown in SEQ ID NO: 20, and a light chain having an amino acid sequence shown in SEQ ID NO: 13;
(h) a heavy chain having an amino acid sequence shown in SEQ ID NO: 21, and a light chain having an amino acid sequence shown in SEQ ID NO: 13;
(i) a heavy chain having an amino acid sequence shown in SEQ ID NO: 22, and a light chain having an amino acid sequence shown in SEQ ID NO: 13;
(j) a heavy chain having an amino acid sequence shown in SEQ ID NO: 23, and a light chain having an amino acid sequence shown in SEQ ID NO: 13;
(k) a heavy chain having an amino acid sequence shown in SEQ ID NO: 24, and a light chain having an amino acid sequence shown in SEQ ID NO: 13;
(l) a heavy chain having an amino acid sequence shown in SEQ ID NO: 27, and a light chain having an amino acid sequence shown in SEQ ID NO: 13; or
(m) a heavy chain having an amino acid sequence shown in SEQ ID NO: 28, and a light chain having an amino acid sequence shown in SEQ ID NO: 13.

In preferable embodiments, the bispecific antibody comprises:
(a) a heavy chain having an amino acid sequence shown in SEQ ID NO: 14, and a light chain having an amino acid sequence shown in SEQ ID NO: 13;
(b) a heavy chain having an amino acid sequence shown in SEQ ID NO: 15, and a light chain having an amino acid sequence shown in SEQ ID NO: 13;
(c) a heavy chain having an amino acid sequence shown in SEQ ID NO: 17, and a light chain having an amino acid sequence shown in SEQ ID NO: 13;
(d) a heavy chain having an amino acid sequence shown in SEQ ID NO: 12, and a light chain having an amino acid sequence shown in SEQ ID NO: 16;
(e) a heavy chain having an amino acid sequence shown in SEQ ID NO: 27, and a light chain having an amino acid sequence shown in SEQ ID NO: 13; or
(f) a heavy chain having an amino acid sequence shown in SEQ ID NO: 28, and a light chain having an amino acid sequence shown in SEQ ID NO: 13.

In some embodiments, the complete anti-5T4 antibody comprises a heavy chain having an amino acid sequence shown in SEQ ID NO: 29, and a light chain having an amino acid sequence shown in SEQ ID NO: 30.

In some embodiments, the bispecific antibody comprises a heavy chain having an amino acid sequence shown in SEQ ID NO: 31, and a light chain having an amino acid sequence shown in SEQ ID NO: 30.

### Immunoconjugates

Another aspect of the disclosure relates to an immunoconjugate, a pharmaceutically acceptable salt thereof, or a solvate thereof, comprising a single domain antibody according to the “Anti-CD16A Single Domain Antibodies” section described above, a heavy chain antibody according to the “Anti-CD16A Heavy Chain Antibodies” section described above, or a bispecific antibody according to the “Bispecific Antibodies” section described above; and an active moiety conjugated to the single domain antibody.

In some embodiments, the active moiety is selected from a group consisting of a toxin, a peptide tag, sortag, a radionuclide, a near-infrared fluorochromes, and a nanoparticle.

In some embodiments, the toxin is selected from the group consisting of a cytotoxic agent, a cytokine, a nucleic acid, a nucleic acid-associated molecule, a radionuclide, a chemokine, aco-stimulatory molecule, an immunosuppressive molecule, a death ligand, an apoptosis-inducing protein, a kinase, a prodrug-converting enzyme, a Rnase, an agonistic antibody or antibody fragment, an antagonistic antibody or antibody fragment, a growth factor, a hormone, a coagulation factor, a fibrinolytic protein, peptides mimicking these, and fragments, fusion proteins and derivatives thereof.

In some embodiments, the radionuclide comprises: At²¹¹, I¹³¹, I¹²⁵, I¹²³, Y⁹⁰, Re¹⁸⁶, Re¹⁸⁸, Sm¹⁵³, Bi²¹², P³², Pb²¹², Tc⁹⁹, S³⁵, F¹⁹, N¹⁵, C¹⁴, C¹³ or H³, optionally the radionuclide can be conjugated to the antibody via a chelating agent.

In some embodiments, provided is an immunoconjugate, a pharmaceutically acceptable salt thereof, or a solvate thereof, comprising a single domain antibody comprising:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1;
and an active moiety conjugated to the single domain antibody, wherein the active moiety is selected from a group consisting of a toxin, a peptide tag, sortag, a radionuclide, a near-infrared fluorochromes, and a nanoparticle.

In some embodiments, provided is an immunoconjugate, a pharmaceutically acceptable salt thereof, or a solvate thereof, comprising a single domain antibody comprising an amino acid sequence selected from a group consisting of SEQ ID NO: 3, 4, 5, 6, 7, 8, 9, 10, 39, and 40; and an active moiety conjugated to the single domain antibody, wherein the active moiety is selected from a group consisting of a toxin, a peptide tag, sortag, a radionuclide, a near-infrared fluorochromes, and a nanoparticle.

In some embodiments, provided is an immunoconjugate, a pharmaceutically acceptable salt thereof, or a solvate thereof, comprising a single domain antibody comprising an amino acid sequence selected from a group consisting of SEQ ID NO: 3, 4, 5, 6, 7, 8, 9, 10, 39, and 40; and an active moiety conjugated to the single domain antibody, wherein the active moiety is a toxin, a radionuclide, or a near-infrared fluorochromes.

In some embodiments, provided is an immunoconjugate, a pharmaceutically acceptable salt thereof, or a solvate thereof, comprising a single domain antibody comprising an amino acid sequence selected from a group consisting of SEQ ID NO: 3, 4, 5, 6, 7, 8, 9, 10, 39, and 40; and a cytotoxic agent conjugated to the single domain antibody.

In some embodiments, provided is an immunoconjugate, a pharmaceutically acceptable salt thereof, or a solvate thereof, comprising a single domain antibody comprising an amino acid sequence selected from a group consisting of SEQ ID NO: 3, 4, 5, 6, 7, 8, 9, 10, 39, and 40; and a radionuclide agent conjugated to the single domain antibody.

### Pharmaceutical Compositions

The present disclosure, in another aspect, provides a pharmaceutical composition comprising the single domain antibody, the heavy chain antibody, the bispecific antibody or the immunoconjugate (hereinafter “antibody or immunoconjugate”) as disclosed herein, and a pharmaceutically acceptable carrier. The compositions are suitable for veterinary or human administration.

The present compositions can be in any form that allows for the composition to be administered to a patient. For example, the composition can be in the form of a solid, liquid or gas (aerosol). Typical routes of administration include, without limitation, oral, topical, parenteral, sublingual, rectal, vaginal, ocular, intra-tumor, and intranasal. Parenteral administration includes subcutaneous injections, intravenous, intramuscular, intrasternal injection or infusion techniques. In one aspect, the compositions are administered parenterally. In yet another aspect, the compositions are administered intravenously.

Pharmaceutical compositions can be formulated so as to allow the active ingredient to be bioavailable upon administration of the composition to a patient. Compositions can take the form of one or more dosage units, where for example, a tablet can be a single dosage unit, and a container of an immunoconjugate in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the immunoconjugate, the manner of administration, and the composition employed.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous or particulate, so as to provide an aerosol composition useful in, e.g., inhalatory administration.

The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of the antibody or immunoconjugate that is effective in the treatment of a particular disorder or condition will depend on the nature of the disorder or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of an antibody or a immunoconjugate such that a suitable dosage will be obtained. Typically, this amount is at least about 0.01% of an antibody or an immunoconjugate by weight of the composition. When intended for oral administration, this amount can be varied to range from about 0.1% to about 80% by weight of the composition. In one aspect, oral compositions can comprise from about 4% to about 50% of the antibody or the immunoconjugate by weight of the composition. In yet another aspect, present compositions are prepared so that a parenteral dosage unit contains from about 0.01% to about 2% by weight of the antibody or the immunoconjugate.

For intravenous administration, the composition can comprise from about 0.01 to about 100 mg of an antibody or an immunoconjugate per kg of the animal's body weight. In one aspect, the composition can include from about 1 to about 100 mg of an antibody or an immunoconjugate per kg of the animal's body weight. In another aspect, the amount administered will be in the range from about 0.1 to about 25 mg/kg of body weight of the antibody or the immunoconjugate.

Generally, the dosage of an antibody or an immunoconjugate administered to a patient is typically about 0.01 mg/kg to about 2000 mg/kg of the animal's body weight. In one aspect, the dosage administered to a patient is between about 0.01 mg/kg to about 10 mg/kg of the animal's body weight, in another aspect, the dosage administered to a patient is between about 0.1 mg/kg and about 250 mg/kg of the animal's body weight, in yet another aspect, the dosage administered to a patient is between about 0.1 mg/kg and about 20 mg/kg of the animal's body weight, in yet another aspect the dosage administered is between about 0.1 mg/kg to about 10 mg/kg of the animal's body weight, and in yet another aspect, the dosage administered is between about 1 mg/kg to about 10 mg/kg of the animal's body weight.

The antibodies, the immunoconjugates or compositions can be administered by any convenient route, for example by infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, capsules, etc., and can be used to administer an antibody or an immunoconjugate or composition. In certain embodiments, more than one antibody, immunoconjugate or composition is administered to a patient.

In specific embodiments, it can be desirable to administer one or more antibodies, immunoconjugates or compositions locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as silastic membranes, or fibers. In one embodiment, administration can be by direct injection at the site (or former site) of a cancer, tumor or neoplastic or pre-neoplastic tissue. In another embodiment, administration can be by direct injection at the site (or former site) of a manifestation of an autoimmune disease.

In certain embodiments, it can be desirable to introduce one or more antibody, immunoconjugate or compositions into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

In yet another embodiment, the antibody, immunoconjugate or compositions can be delivered in a controlled release system, such as but not limited to, a pump or various polymeric materials can be used. In yet another embodiment, a controlled-release system can be placed in proximity of the target of the antibody, immunoconjugate or compositions, e.g., the brain, thus requiring only a fraction of the systemic dose.

The term “carrier” refers to a diluent, adjuvant or carrier, with which an antibody or a immunoconjugate is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a patient, the antibody or the immunoconjugate or compositions and pharmaceutically acceptable carriers are sterile. Water is an exemplary carrier when the antibodies or immunoconjugates are administered intravenously. Saline solutions and aqueous dextrose and glycerol solutions can also be employed as liquid carriers, particularly for injectable solutions. Suitable pharmaceutical carriers also include carriers such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

In an embodiment, the immunoconjugates are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette Indicating the quantity of active agent. Where a immunoconjugate is to be administered by Infusion, It can be dispensed, for example, with an infusion bottle containing sterile pharmaceutical grade water or saline. Where the antibody or the immunoconjugate is administered by injection, an ampoule of sterile water for injection or saline can be provided so that the ingredients can be mixed prior to administration.

The composition can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients.

Whether in solid, liquid or gaseous form, the present compositions can include a pharmacological agent used in the treatment of cancer.

### Uses and Therapies

The antibodies and immunoconjugates are useful for inhibiting the multiplication of a tumor cell or cancer cell, causing apoptosis in a tumor or cancer cell, or for treating cancer in a patient. The antibodies and the immunoconjugates can be used accordingly in a variety of settings for the treatment of animal cancers. The antibodies and immunoconjugates can be used to deliver a drug or drug unit to a tumor cell or cancer cell. Without being bound by theory, in one embodiment, the antibody or the antibody moiety of the immunoconjugate disclosed herein binds to or associates with CD16A expressed on, or associated with, the NK cell surface. In the case of a bispecific antibody, the antibody binds to CD16A with one arm and to a second antigen expressed on a tumor cell via the other arm and forms a “NK engager” to enhance the function of NK cells to kill the tumor cells.

The present disclosure, in a further aspect, provides a method for treating cancer in a subject, comprising administering to the subject a therapeutically effective amount of the single domain antibody, the heavy chain antibody, the bispecific antibody or the immunoconjugate disclosed herein.

The present disclosure, in a further aspect, provides a method for prevention of cancer in a subject at a risk of suffering from the cancer, comprising administering to the subject a prophylactically effective amount of the single domain antibody, the heavy chain antibody, the bispecific antibody or the immunoconjugate disclosed herein.

The present disclosure, in a further aspect, provides a method for diagnosing, for example in vitro, a cancer in a subject, comprising isolating a sample from the subject; contacting, for example in vitro, the sample with the single domain antibody, the heavy chain antibody, the bispecific antibody or the immunoconjugate disclosed herein; detecting a signal generated by the contacting; comparing the signal with a threshold; and determining whether the subject is suffered from the cancer based on an outcome of the comparation.

The cancer may be a solid cancer or a hematologic malignancy. A “hematologic malignancy”, also known as a blood cancer, is a cancer that originates in blood-forming tissue, such as the bone marrow or other cells of the immune system. Hematologic malignancies include, without limitation, leukemias (such as acute myeloid leukemia (ANIL), acute promyelocytic leukemia, acute lymphoblastic leukemia (ALL), acute mixed lineage leukemia, chronic myeloid leukemia, chronic lymphocytic leukemia (CLL), hairy, cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies. “Solid cancers” include, without limitation, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma. Preferably, the cancers treatable by the present disclosure include breast cancer.

In some embodiments, provided are methods for treating or preventing cancer are provided, including administering to a patient in need thereof a therapeutically effective amount of an antibody or an immunoconjugate disclosed herein and a chemotherapeutic agent. In one embodiment, the chemotherapeutic agent is that with which treatment of the cancer has not been found to be refractory. In another embodiment, the chemotherapeutic agent is that with which the treatment of cancer has been found to be refractory. The antibodies or the immunoconjugates can be administered to a patient that has also undergone surgery as treatment for the cancer. In some embodiments, the patient has been found to be refractory to a HER2 antibody, for example, Trastuzumab or Margetuximab.

In a specific embodiment, the antibody or the immunoconjugate is administered concurrently with the chemotherapeutic agent or with radiation therapy. In another specific embodiment, the chemotherapeutic agent or radiation therapy is administered prior or subsequent to administration of an antibody or an immunoconjugate, in one aspect at least an hour, five hours, 12 hours, a day, a week, a month, in further aspects several months (e.g., up to three months), prior or subsequent to administration of an antibody or a immunoconjugate.

A chemotherapeutic agent can be administered over a series of sessions. Suitable chemotherapeutic agents include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, 47 ytoxan 47 zine, topotecan, nitrogen mustards, 47ytoxan, etoposide, 5-fluorouracil, BCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. With respect to radiation, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, methods of treatment of cancer with an antibody or an immunoconjugate disclosed herein are provided as an alternative to chemotherapy or radiation therapy where the chemotherapy or the radiation therapy has proven or can prove too toxic, e.g., results in unacceptable or unbearable side effects, for the subject being treated. The animal being treated can, optionally, be treated with another cancer treatment such as surgery, radiation therapy or chemotherapy, depending on which treatment is found to be acceptable or bearable.

The antibodies or the immunoconjugates can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the animal's remaining bone-marrow cell population is then eradicated via the administration of a high dose of an antibody or a immunoconjugate with or without accompanying high dose radiation therapy, and the stem cell graft is infused back into the animal. Supportive care is then provided while bone marrow function is restored and the animal recovers.

Equivalently, the present disclosure also provides the single domain antibody, the heavy chain antibody, the bispecific antibody or the immunoconjugate as disclosed herein for use in the treatment of cancer in a subject. Equivalently, the present disclosure also provides use of the single domain antibody, the heavy chain antibody, the bispecific antibody or the immunoconjugate as disclosed herein in the manufacturing of a medicament for treatment of cancer in a subject.

The present disclosure also provides a method for treatment of acute or chronic infection, for example infection caused by virus, fungi or bacteria, comprising administering to a subject in need thereof a therapeutically effective amount of the single domain antibody, the heavy chain antibody, the bispecific antibody or the immunoconjugate as disclosed herein. For example, the infection to be treated by the single domain antibody, the heavy chain antibody, the bispecific antibody or the immunoconjugate as disclosed herein may be infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenza virus), a paramyxovirus (e.g., parainfluenza virus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; or other infectious diseases, such as chlamydia, fungal diseases including but not limited to candidiasis, aspergillosis, histoplasmosis, cryptococcal meningitis, or parasitic diseases including but not limited to malaria, Pneumocystis carinii pneumonia, leishmaniasis, cryptosporidiosis, toxoplasmosis, and trypanosome infection.

The present disclosure also provides a method for treatment of aging-associated ailment or disease, comprising administering to a subject in need thereof a therapeutically effective amount of the single domain antibody, the heavy chain antibody, the bispecific antibody or the immunoconjugate as disclosed herein. For example, the aging-associated disease is any of atherosclerosis, cardiovascular disease, arthritis, cataracts, osteoporosis, type 2 diabetes, hypertension and Alzheimer's disease.

Equivalently, the present disclosure also provides the single domain antibody, the heavy chain antibody, the bispecific antibody or the immunoconjugate as disclosed herein for use in the treatment of acute or chronic infection in a subject. Equivalently, the present disclosure also provides use of the single domain antibody, the heavy chain antibody, the bispecific antibody or the immunoconjugate as disclosed herein in the manufacturing of a medicament for treatment of acute or chronic infection in a subject.

Equivalently, the present disclosure also provides the single domain antibody, the heavy chain antibody, the bispecific antibody or the immunoconjugate as disclosed herein for use in the treatment of aging-associated ailment in a subject. Equivalently, the present disclosure also provides use of the single domain antibody, the heavy chain antibody, the bispecific antibody or the immunoconjugate as disclosed herein in the manufacturing of a medicament for treatment of aging-associated ailment in a subject.

The present disclosure, in a further aspect, provides a method for prevention of acute or chronic infection in a subject at a risk of suffering from the acute or chronic infection, comprising administering to the subject a prophylactically effective amount of the single domain antibody, the heavy chain antibody, the bispecific antibody or the immunoconjugate disclosed herein.

The present disclosure, in a further aspect, provides a method for prevention of aging-associated ailment in a subject at a risk of suffering from the aging-associated ailment, comprising administering to the subject a prophylactically effective amount of the single domain antibody, the heavy chain antibody, the bispecific antibody or the immunoconjugate disclosed herein.

The present disclosure, in a further aspect, provides a method for diagnosing, for example in vitro, acute or chronic infection in a subject, comprising isolating a sample from the subject; contacting, for example in vitro, the sample with the single domain antibody, the heavy chain antibody, the bispecific antibody or the immunoconjugate disclosed herein; detecting a signal generated by the contacting; comparing the signal with a threshold; and determining whether the subject is suffered from the acute or chronic infection based on an outcome of the comparation.

The present disclosure, in a further aspect, provides a method for diagnosing, for example in vitro, an aging-associated ailment in a subject, comprising isolating a sample from the subject; contacting, for example in vitro, the sample with the single domain antibody, the heavy chain antibody, the bispecific antibody or the immunoconjugate disclosed herein; detecting a signal generated by the contacting; comparing the signal with a threshold; and determining whether the subject is suffered from the aging-associated ailment based on an outcome of the comparation.

The present disclosure, in a further aspect, provides a method for detecting CD16A positive sample in vitro, comprising contacting in vitro the sample with the single domain antibody, the heavy chain antibody, the bispecific antibody or the immunoconjugate disclosed herein; detecting a signal generated by the contacting; comparing the signal with a threshold; and determining whether the sample is positive for CD16A.

Sequences

| SEQ ID | | |
|---|---|---|
| NO: | Description | |
| (Sequence | (Sequence | |
| Number) | Name) | Amino Acid Sequences |
| 1 | BM156-01 | EVQLVESGGGLVQPGGSLRLSCAASGFTFSNYGMSW |
| | | VRQAPGKGLEWIGSIYYSGSTNYNPSLKSLVTISRDNS |
| | | KNTLYLQMNSLRAEDTATYYCARESIDYWGQGTLVT |
| | | VSS |
| 2 | Human CD16A | GMRTEDLPKAVVFLEPQWYRVLEKDSVTLKCQGAYS |
| | (158V) | PEDNSTQWFHNESLISSQASSYFIDAATVDDSGEYRCQ |
| | | TNLSTLSDPVQLEVHIGWLLLQAPRWVFKEEDPIHLR |
| | | CHSWKNTALHKVTYLQNGKGRKYFHHNSDFYIPKAT |
| | | LKDSGSYFCRGLVGSKNVSSETVNITITQGLAVSTISSF |
| | | FPPGYQ |
| 3 | CD16-VH- | EVQLVESGGGLVQPGGSLRLSCAASGFTFSNYGMSW |
| | RM72 | VRQAPGKGLEWIGSIYYSGQTNYNPSLKSLVTISRDNS |
| | | KNTLYLQMNSLRAEDTATYYCARESIDYWGQGTLVT |
| | | VSS |
| 4 | CD16-VH- | EVQLVESGGGLVQPGGSLRLSCAASGFTFSNYGMSW |
| | RM73 | VRQAPGKGLEWIGSIYYSGTTNYNPSLKSLVTISRDNS |
| | | KNTLYLQMNSLRAEDTATYYCARESIDYWGQGTLVT |
| | | VSS |
| 5 | CD16-VH- | EVQLVESGGGLVQPGGSLRLSCAASGFTFSNYGMSW |
| | RM75 | VRQAPGKGLEWIGSIYYSGDTNYNPSLKSLVTISRDNS |
| | | KNTLYLQMNSLRAEDTATYYCARESIDYWGQGTLVT |
| | | VSS |
| 6 | CD16-VH- | EVQLVESGGGLVQPGGSLRLSCAASGFTFSNYGMSW |
| | RM76 | VRQAPGKGLEWIGSIYYSGETNYNPSLKSLVTISRDNS |
| | | KNTLYLQMNSLRAEDTATYYCARESIDYWGQGTLVT |
| | | VSS |
| 7 | CD16-VH- | EVQLVESGGGLVQPGGSLRLSCAASGFTFSNYGMSW |
| | RM77 | VRQAPGKGLEWIGSIYYNGSTNYNPSLKSLVTISRDNS |
| | | KNTLYLQMNSLRAEDTATYYCARESIDYWGQGTLVT |
| | | VSS |
| 8 | CD16-VH- | EVQLVESGGGLVQPGGSLRLSCAASGFTFSNYGMSW |
| | RM80 | VRQAPGKGLEWIGSIYYDGSTNYNPSLKSLVTISRDNS |
| | | KNTLYLQMNSLRAEDTATYYCARESIDYWGQGTLVT |
| | | VSS |
| 9 | CD16-VH- | EVQLVESGGGLVQPGGSLRLSCAASGFTFSNYGMSW |
| | RM81 | VRQAPGKGLEWIGSIYYTGSTNYNPSLKSLVTISRDNS |
| | | KNTLYLQMNSLRAEDTATYYCARESIDYWGQGTLVT |
| | | VSS |
| 10 | CD16-VH- | EVQLVESGGGLVQPGGSLRLSCAASGFTFSNYGMSW |
| | RM82 | VRQAPGKGLEWIGSIYYSVSTNYNPSLKSLVTISRDNS |
| | | KNTLYLQMNSLRAEDTATYYCARESIDYWGQGTLVT |
| | | VSS |
| 11 | Human CD16A | GMRTEDLPKAVVFLEPQWYRVLEKDSVTLKCQGAYS |
| | (158F) | PEDNSTQWFHNESLISSQASSYFIDAATVDDSGEYRCQ |
| | | TNLSTLSDPVQLEVHIGWLLLQAPRWVFKEEDPIHLR |
| | | CHSWKNTALHKVTYLQNGKGRKYFHHNSDFYIPKAT |
| | | LKDSGSYFCRGLFGSKNVSSETVNITITQGLAVSTISSF |
| | | FPPGYQ |
| 12 | BM130-93: | EVQLVESGGGLVQPGGSLRLSCAASGFNIKDTYIHWV |
| | Trastuzumab | RQAPGKGLEWVARIYPTNGYTRYADSVKGRFTISADT |
| | Heavy chain | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGK |
| 13 | BM130-93: | DIQMTQSPSSLSASVGDRVTITCRASQDVNTAVAWYQ |
| | Trastuzumab | QKPGKAPKLLIYSASFLYSGVPSRFSGSRSGTDFTLTIS |
| | Light chain | SLQPEDFATYYCQQHYTTPPTFGQGTKVEIKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| 14 | BMP01-16 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGQTNYNPSLKSLVTISRDNSKNTLYLQMN |
| | | SLRAEDTATYYCARESIDYWGQGTLVTVSS |
| 15 | BMP01-16b | EVQLVESGGGLVQPGGSLRLSCAASGFNIKDTYIHWV |
| | HC ((G4S)₄) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSGGGGSEVQLV |
| | | ESGGGLVQPGGSLRLSCAASGFTFSNYGMSWVRQAP |
| | | GKGLEWIGSIYYSGQTNYNPSLKSLVTISRDNSKNTLY |
| | | LQMNSLRAEDTATYYCARESIDYWGQGTLVTVSS |
| 16 | BMP01-16c | DIQMTQSPSSLSASVGDRVTITCRASQDVNTAVAWYQ |
| | LC: | QKPGKAPKLLIYSASFLYSGVPSRFSGSRSGTDFTLTIS |
| | Trastuzumab | SLQPEDFATYYCQQHYTTPPTFGQGTKVEIKRTVAAP |
| | Light chain | SVFIFPPSDEQLKSGTASVVCLLNNFYPREAKVQWKV |
| | (G4S)₃ CD16A | DNALQSGNSQESVTEQDSKDSTYSLSSTLTLSKADYE |
| | | KHKVYACEVTHQGLSSPVTKSFNRGECGGGGSGGGG |
| | | SGGGGSEVQLVESGGGLVQPGGSLRLSCAASGFTFSN |
| | | YGMSWVRQAPGKGLEWIGSIYYSGQTNYNPSLKSLV |
| | | TISRDNSKNTLYLQMNSLRAEDTATYYCARESIDYWG |
| | | QGTLVTVSS |
| 17 | BMP01-16d | EVQLVESGGGLVQPGGSLRLSCAASGFTFSNYGMSW |
| | HC: CD16A | VRQAPGKGLEWIGSIYYSGQTNYNPSLKSLVTISRDNS |
| | (G4S)₃ | KNTLYLQMNSLRAEDTATYYCARESIDYWGQGTLVT |
| | Trastuzumab | VSSGGGGSGGGGSGGGGSGGGGSEVQLVESGGGLVQ |
| | Heavy chain | PGGSLRLSCAASGFNIKDTYIHWVRQAPGKGLEWVA |
| | | RIYPTNGYTRYADSVKGRFTISADTSKNTAYLQMNSL |
| | | RAEDTAVYYCSRWGGDGFYAMDYWGQGTLVTVSS |
| | | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSCDKTHTCPPCP |
| | | APELLGGPSVFLFPPKPKDTLMISRTPEVTCVVVDVSH |
| | | EDPEVKFNWYVDGVEVHNAKTKPREEQYNSTYRVV |
| | | SVLTVLHQDWLNGKEYKCKVSNKALPAPIEKTISKAK |
| | | GQPREPQVYTLPPSREEMTKNQVSLTCLVKGFYPSDI |
| | | AVEWESNGQPENNYKTTPPVLDSDGSFFLYSKLTVDK |
| | | SRWQQGNVFSCSVMHEALHNHYTQKSLSLSPGK |
| 18 | BMP01-17 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGTTNYNPSLKSLVTISRDNSKNTLYLQMNS |
| | | LRAEDTATYYCARESIDYWGQGTLVTVSS |
| 19 | BMP01-18 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGDTNYNPSLKSLVTISRDNSKNTLYLQMN |
| | | SLRAEDTATYYCARESIDYWGQGTLVTVSS |
| 20 | BMP01-19 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYNGSTNYNPSLKSLVTISRDNSKNTLYLQMN |
| | | SLRAEDTATYYCARESIDYWGQGTLVTVSS |
| 21 | BMP01-20 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGSTNYNPSLKSLVTISRDNSKNTLYLQMNS |
| | | LRAEDTATYYCARESIDYWGQGTLVTVSS |
| 22 | BMP01-21 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYTGSTNYNPSLKSLVTISRDNSKNTLYLQMNS |
| | | LRAEDTATYYCARESIDYWGQGTLVTVSS |
| 23 | BMP01-22 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGGGGGSGGGGSEVQLVESGGG |
| | | LVQPGGSLRLSCAASGFTFSNYGMSWVRQAPGKGLE |
| | | WIGSIYYNGQTNYNPSLKSLVTISRDNSKNTLYLQMN |
| | | SLRAEDTATYYCARESIDYWGQGTLVTVSS |
| 24 | BMP01-23 HC | EVQLVESGGGLVQPGGSLRLSCAASGFNIKDTYIHWV |
| | ((G4S)₃) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYTGQTNYNPSLKSLVTISRDNSKNTLYLQMN |
| | | SLRAEDTATYYCARESIDYWGQGTLVTVSS |
| 25 | BM130-90: | EVQLVESGGGLVQPGGSLRLSCAASGFNIKDTYIHWV |
| | Trastuzumab | RQAPGKGLEWVARIYPTNGYTRYADSVKGRFTISADT |
| | HC L234A | SKNTAYLQMNSLRAEDTAVYYCSRWGGDGFYAMDY |
| | L235A | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGK |
| 26 | BM130-91: | EVQLVESGGGLVQPGGSLRLSCAASGFNIKDTYIHWV |
| | Trastuzumab | RQAPGKGLEWVARIYPTNGYTRYADSVKGRFTISADT |
| | HC N297A | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYASTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGK |
| 27 | BMP01-24 HC | EVQLVESGGGLVQPGGSLRLSCAASGFNIKDTYIHWV |
| | (L234A | RQAPGKGLEWVARIYPTNGYTRYADSVKGRFTISADT |
| | L235A) | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPEAAGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGQTNYNPSLKSLVTISRDNSKNTLYLQMN |
| | | SLRAEDTATYYCARESIDYWGQGTLVTVSS |
| 28 | BMP01-25 HC | EVQLVESGGGLVQPGGSLRLSCAASGFNIKDTYIHWV |
| | (N297A) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKTK |
| | | PREEQYASTYRVVSVLTVLHQDWLNGKEYKCKVSN |
| | | KALPAPIEKTISKAKGQPREPQVYTLPPSREEMTKNQV |
| | | SLTCLVKGFYPSDIAVEWESNGQPENNYKTTPPVLDS |
| | | DGSFFLYSKLTVDKSRWQQGNVFSCSVMHEALHNHY |
| | | TQKSLSLSPGKGGGGSGGGGSGGGGSEVQLVESGGG |
| | | LVQPGGSLRLSCAASGFTFSNYGMSWVRQAPGKGLE |
| | | WIGSIYYSGQTNYNPSLKSLVTISRDNSKNTLYLQMN |
| | | SLRAEDTATYYCARESIDYWGQGTLVTVSS |
| 29 | Anti-5T4 | EVQLVQSGGGVVRPGGSLRLSCAGSGFNFDDYGMSW |
| | antibody m603 | VRQAPGKGLEWVSGINWNGGSTGYADSVKGRFTISR |
| | Heavy chain | DNAKNSLYLQMNSLRAEDTALYYCARGAFWNSGSG |
| | | NSPDYWGQGTLVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| 30 | Anti-5T4 | DIQMTQSPSSLSASVGDRVTITCRASQDVNTAVAWYQ |
| | antibody m603 | QKPGKAPKLLIYSASFLYSGVPSRFSGSRSGTDFTLTIS |
| | Light chain | SLQPEDFATYYCQQHYTTPPTFGQGTKVEIKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| 31 | BMP02-10 | EVQLVQSGGGVVRPGGSLRLSCAGSGFNFDDYGMSW |
| | Heavy chain | VRQAPGKGLEWVSGINWNGGSTGYADSVKGRFTISR |
| | | DNAKNSLYLQMNSLRAEDTALYYCARGAFWNSGSG |
| | | NSPDYWGQGTLVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGKGGGGSGGGGSGGGGSEVQLVE |
| | | SGGGLVQPGGSLRLSCAASGFTFSNYGMSWVRQAPG |
| | | KGLEWIGSIYYSGQTNYNPSLKSLVTISRDNSKNTLYL |
| | | QMNSLRAEDTATYYCARESIDYWGQGTLVTVSS |
| 32 | human HER2 | TQVCTGTDMKLRLPASPETHLDMLRHLYQGCQVVQ |
| | (NP_004439.2\| | GNLELTYLPTNASLSFLQDIQEVQGYVLIAHNQVRQV |
| | 23-652) | PLQRLRIVRGTQLFEDNYALAVLDNGDPLNNTTPVTG |
| | | ASPGGLRELQLRSLTEILKGGVLIQRNPQLCYQDTILW |
| | | KDIFHKNNQLALTLIDTNRSRACHPCSPMCKGSRCWG |
| | | ESSEDCQSLTRTVCAGGCARCKGPLPTDCCHEQCAA |
| | | GCTGPKHSDCLACLHFNHSGICELHCPALVTYNTDTF |
| | | ESMPNPEGRYTFGASCVTACPYNYLSTDVGSCTLVCP |
| | | LHNQEVTAEDGTQRCEKCSKPCARVCYGLGMEHLRE |
| | | VRAVTSANIQEFAGCKKIFGSLAFLPESFDGDPASNTA |
| | | PLQPEQLQVFETLEEITGYLYISAWPDSLPDLSVFQNL |
| | | QVIRGRILHNGAYSLTLQGLGISWLGLRSLRELGSGLA |
| | | LIHHNTHLCFVHTVPWDQLFRNPHQALLHTANRPEDE |
| | | CVGEGLACHQLCARGHCWGPGPTQCVNCSQFLRGQE |
| | | CVEECRVLQGLPREYVNARHCLPCHPECQPQNGSVT |
| | | CFGPEADQCVACAHYKDPPFCVARCPSGVKPDLSYM |
| | | PIWKFPDEEGACQPCPINCTHSCVDLDDKGCPAEQRA |
| | | SPLT |
| 33 | Monkey | GMRAEDLPKAVVFLEPQWYRVLEKDRVTLKCQGAY |
| | CD16A | SPEDNSTRWFHNESLISSQTSSYFIAAARVNNSGEYRC |
| | | QTSLSTLSDPVQLEVHIGWLLLQAPRWVFKEEESIHLR |
| | | CHSWKNTLLHKVTYLQNGKGRKYFHQNSDFYIPKAT |
| | | LKDSGSYFCRGLIGSKNVSSETVNITITQDLAVSSISSFF |
| | | PPGYQ |
| 34 | Mouse CD16A | LPKAVVKLDPPWIQVLKEDMVTLMCEGTHNPGNSST |
| | | QWFHNGRSVRSQVQASYTFKATVNDSGEYRCQMEQ |
| | | TRLSDPVDLGVISDWLLLQTPQRVFLEGETITLRCHSW |
| | | RNKLLNRISFFHNEKSVRYHHYKSNFSIPKANHSHSG |
| | | DYYCKGSLGSTQHQSKPVTITVQDPATTSSISLVWYH |
| | | T |
| 35 | Rat CD16A | LQKAVVILDPEWVRVLEEDCVILRCQGTFSPEDNSTK |
| | | WFHNKSLISHQDANYVIQSARVKDSGMYRCQTAFSA |
| | | LSDPVQLDVHADWLLLQTTKRLFQEGDPIRLRCHSW |
| | | RNTPVFKVTYLQNGKGKKYFHRNSELSISKATHADSG |
| | | SYFCRGIIGRNNISSASLQISIGDPTSPSSFLP |
| 36 | Human CD16B | GMRTEDLPKAVVFLEPQWYRVLEKDSVTLKCQGAYS |
| | | PEDNSTQWFHNENLISSQASSYFIDAATVDDSGEYRC |
| | | QTNLSTLSDPVQLEVHVGWLLLQAPRWVFKEEDPIHL |
| | | RCHSWKNTALHKVTYLQNGKDRKYFHHNSDFHIPKA |
| | | TLKDSGSYFCRGLVGSKNVSSETVNITITQGLAVSTIS |
| 37 | BM130-92 | QVQLQQSGPE LVKPGASLKL SCTASGFNIK |
| | Margetuximab | DTYIHWVKQR PEQGLEWIGR IYPTNGYTRY |
| | Heavy chain | DPKFQDKATI TADTSSNTAY LQVSRLTSED |
| | | TAVYYCSRWG GDGFYAMDYW GQGASVTVSS |
| | | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKRVEP KSCDKTHTCP PCPAPELVGG |
| | | PSVFLLPPKP KDTLMISRTP EVTCVVVDVS |
| | | HEDPEVKFNW YVDGVEVHNA KTKPPEEQYN |
| | | STLRVVSVLT VLHQDWLNGK EYKCKVSNKA |
| | | LPAPIEKTIS KAKGQPREPQ VYTLPPSRDE |
| | | LTKNQVSLTC LVKGFYPSDI AVEWESNGQP |
| | | ENNYKTTPLV LDSDGSFFLY SKLTVDKSRW |
| | | QQGNVFSCSV MHEALHNHYT QKSLSLSPGK |
| 38 | BM130-92 | DIVMTQSHKF MSTSVGDRVS ITCKASQDVN |
| | Margetuximab | TAVAWYQQKP GHSPKLLIYS ASFRYTGVPD |
| | Light chain | RFTGSRSGTD FTFTISSVQA EDLAVYYCQQ |
| | | HYTTPPTFGG GTKVEIKRTV AAPSVFIFPP |
| | | SDEQLKSGTA SVVCLLNNFY PREAKVQWKV |
| | | DNALQSGNSQ ESVTEQDSKD STYSLSSTLT |
| | | LSKADYEKHK VYACEVTHQG LSSPVTKSFN RGEC |
| 39 | BMP01-22 HC | EVQLVESGGGLVQPGGSLRLSCAASGFTFSNYGMSW |
| | VHH | VRQAPGKGLEWIGSIYYNGQTNYNPSLKSLVTISRDN |
| | | SKNTLYLQMNSLRAEDTATYYCARESIDYWGQGTLV |
| | | TVSS |
| 40 | BMP01-23 HC | EVQLVESGGGLVQPGGSLRLSCAASGFTFSNYGMSW |
| | VHH | VRQAPGKGLEWIGSIYYTGQTNYNPSLKSLVTISRDNS |
| | | KNTLYLQMNSLRAEDTATYYCARESIDYWGQGTLVT |
| | | VSS |
| 41 | CDR1-IMGT | GFTFSNYG |
| 42 | CDR3-IMGT | ARESIDY |
| 43 | CDR2-IMGT | IYYSGQT |
| 44 | CDR2-IMGT | IYYSGTT |
| 45 | CDR2-IMGT | IYYSGDT |
| 46 | CDR2-IMGT | IYYSGET |
| 47 | CDR2-IMGT | IYYNGST |
| 48 | CDR2-IMGT | IYYDGST |
| 49 | CDR2-IMGT | IYYTGST |
| 50 | CDR2-IMGT | IYYSVST |
| 51 | CDR2-IMGT | IYYNGQT |
| 52 | CDR2-IMGT | IYYTGQT |
| 53 | CDR1-Kabat | NYGMS |
| 54 | CDR3-Kabat | ESIDY |
| 55 | CDR2-Kabat | SIYYSGQTNYNPSLKS |
| 56 | CDR2-Kabat | SIYYSGTTNYNPSLKS |
| 57 | CDR2-Kabat | SIYYSGDTNYNPSLKS |
| 58 | CDR2-Kabat | SIYYSGETNYNPSLKS |
| 59 | CDR2-Kabat | SIYYNGSTNYNPSLKS |
| 60 | CDR2-Kabat | SIYYDGSTNYNPSLKS |
| 61 | CDR2-Kabat | SIYYTGSTNYNPSLKS |
| 62 | CDR2-Kabat | SIYYSVSTNYNPSLKS |
| 63 | CDR2-Kabat | SIYYNGQTNYNPSLKS |
| 64 | CDR2-Kabat | SIYYTGQTNYNPSLKS |
| 65 | CDR1-Chothia | GFTFSNY |
| 66 | CDR3-Chothia | ESIDY |
| 67 | CDR2-Chothia | YYSGQ |
| 68 | CDR2-Chothia | YYSGT |
| 69 | CDR2-Chothia | YYSGD |
| 70 | CDR2-Chothia | YYSGE |
| 71 | CDR2-Chothia | YYNGS |
| 72 | CDR2-Chothia | YYDGS |
| 73 | CDR2-Chothia | YYTGS |
| 74 | CDR2-Chothia | YYSVS |
| 75 | CDR2-Chothia | YYNGQ |
| 76 | CDR2-Chothia | YYTGQ |
| 77 | CDR1-Contact | SNYGMS |
| 78 | CDR3-Contact | ARESID |
| 79 | CDR2-Contact | WIGSIYYSGQTN |
| 80 | CDR2-Contact | WIGSIYYSGTTN |
| 81 | CDR2-Contact | WIGSIYYSGDTN |
| 82 | CDR2-Contact | WIGSIYYSGETN |
| 83 | CDR2-Contact | WIGSIYYNGSTN |
| 84 | CDR2-Contact | WIGSIYYDGSTN |
| 85 | CDR2-Contact | WIGSIYYTGSTN |
| 86 | CDR2-Contact | WIGSIYYSVSTN |
| 87 | CDR2-Contact | WIGSIYYNGQTN |
| 88 | CDR2-Contact | WIGSIYYTGQTN |
| 89 | Human Fc | EPKSCDKTHTCPPCPAPELLGGPSVFLFPPKPKDTLMIS |
| | Portion | RTPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSREEMTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGK |
| 90 | Human IgG1 | EPKSCDKTHTCPPCPAPELLGGP |
| | Hinge region | |

Note:
where the description relates to CDRs (e.g., SEQ ID NO: 88), the terms IMGT, Kabat, Chothia, or Contact refer to the different CDR definition schemes as described in the Definition section.

### EXAMPLES

The following examples are included to demonstrate preferred embodiments of the invention and should not be used to limit the scope of the invention.

### Example 1. CD16A VH Affinity Maturation

This example illustrates a method of affinity maturation by panning a random mutation phage library. A previously identified CD16A VH BM156-01 (SEQ ID NO: 1) was used as template for random mutation libraries construction. NNB primers were designed for each amino acid in the annotated CDR (as shown in Table 1). Antibody numbering and CDR annotation were performed according to IMGT scheme. PCR was performed to construct an arbitrary mutation library for each amino acid in the CDR region.

TABLE 1
List of mutagenesis primers for library
construction

| | PRIMER | |
|---|---|---|
| SEQ ID NO: | NAME | SEQUENCES (5′ TO 3′) |
| 91 | G26+ | TGCAGCCTCTNNBTTCACCT |
| 92 | G26− | AGGTGAAVNNAGAGGCTGCA |
| 93 | F27+ | TGCAGCCTCTGGANNBACCTTCAGT |
| 94 | F27− | ACTGAAGGTVNNTCCAGAGGCTGCA |
| 95 | T28+ | GCCTCTGGATTCNNBTTCAGTAAC |
| 96 | T28− | GTTACTGAAVNNGAATCCAGAGGC |
| 97 | F29+ | TCTGGATTCACCNNBAGTAACTAT |
| 98 | F29− | ATAGTTACTVNNGGTGAATCCAGA |
| 99 | S30+ | GGATTCACCTTCNNBAACTATGGT |
| 100 | S30− | ACCATAGTTVNNGAAGGTGAATCC |
| 101 | N31+ | TTCACCTTCAGTNBTATGGTATG |
| 102 | N31− | CATACCATAVNNACTGAAGGTGAA |
| 103 | Y32+ | ACCTTCAGTAACNNBGGTATGAGCTGG |
| 104 | Y32− | CCAGCTCATACCVNNGTTACTGAAGGT |
| 105 | G33+ | TTCAGTAACTATNNBATGAGCTGGGT |
| 106 | G33− | ACCCAGCTCATVNNATAGTTACTGAA |
| 107 | I51+ | GATTGGGAGTNNBTATTACAGTGG |
| 108 | I51− | CCACTGTAATAVNNACTCCCAATC |
| 109 | Y52+ | TGGGAGTATCNNBTACAGTGGG |
| 110 | Y52− | CCCACTGTAVNNGATACTCCCA |
| 111 | Y53+ | GAGTATCTATNNBAGTGGGAGCAC |
| 112 | Y53− | GTGCTCCCACTVNNATAGATACTC |
| 113 | S54+ | AGTATCTATTACNNBGGGAGCACCAAC |
| 114 | S54− | GTTGGTGCTCCCVNNGTAATAGATACT |
| 115 | G55+ | ATCTATTACAGTNNBAGCACCAACTAC |
| 116 | G55− | GTAGTTGGTGCTVNNACTGTAATAGAT |
| 117 | S56+ | TATTACAGTGGGNNBACCAACTACAAC |
| 118 | S56− | GTTGTAGTTGGTVNNCCCACTGTAATA |
| 119 | T57+ | TACAGTGGGAGCNNBAACTACAACCCCT |
| 120 | T57− | AGGGGTTGTAGTTVNNGCTCCCACTGTA |
| 121 | 96A+ | GTATTACTGTNNBAGAGAGAGTA |
| 122 | 96A− | TACTCTCTCTVNNACAGTAATAC |
| 123 | 97R+ | TTACTGTGCGNNBGAGAGTATTG |
| 124 | 97R− | CAATACTCTCVNNCGCACAGTAA |
| 125 | 98E+ | CTGTGCGAGANNBAGTATTGACT |
| 126 | 98E− | AGTCAATACTVNNTCTCGCACAG |
| 127 | 99S+ | TGCGAGAGAGNNBATTGACTACT |
| 128 | 99S− | AGTAGTCAATVNNCTCTCTCGCA |
| 129 | 100I+ | GAGAGAGAGTNNBGACTACTGGG |
| 130 | 100I− | CCCAGTAGTCVNNACTCTCTCTC |
| 131 | 101D+ | AGAGAGTATTNNBTACTGGGGCC |
| 132 | 101D− | GGCCCCAGTAVNNAATACTCTCT |
| 133 | 102Y+ | GAGTATTGACNNBTGGGGCCAGG |
| 134 | 102Y− | CCTGGCCCCAVNNGTCAATACTC |
| 91 | 96A+ | GTATTACTGTNNBAGAGAGAGTA |
| 92 | 96A− | TACTCTCTCTVNNACAGTAATAC |
| 93 | 97R+ | TTACTGTGCGNNBGAGAGTATTG |
| 94 | 97R− | CAATACTCTCVNNCGCACAGTAA |
| 95 | 98E+ | CTGTGCGAGANNBAGTATTGACT |
| 96 | 98E− | AGTCAATACTVNNTCTCGCACAG |
| 97 | 99S+ | TGCGAGAGAGNNBATTGACTACT |
| 98 | 99S− | AGTAGTCAATVNNCTCTCTCGCA |
| 99 | 100I+ | GAGAGAGAGTNNBGACTACTGGG |
| 100 | 100I− | CCCAGTAGTCVNNACTCTCTCTC |
| 101 | 101D+ | AGAGAGTATTNNBTACTGGGGCC |
| 102 | 101D− | GGCCCCAGTAVNNAATACTCTCT |
| 103 | 102Y+ | GAGTATTGACNNBTGGGGCCAGG |
| 104 | 102Y− | CCTGGCCCCAVNNGTCAATACTC |
| 105 | RPPP | CAGCTGGCACGACAGGTTTC |
| 106 | G3RRR | TATTAGCGTTTGCCATCTTT |

Phage library panning was performed according to the following procedure. Biotin-labeled recombinant human CD16A protein (hCD16A Seq ID No: 2) was immobilized on streptavidin-coated magnetic beads and beads were washed 3 times with PBS buffer containing 2% BSA, then CD16A-coated beads were incubated with 1010 phage at room temperature for two hours.

After washing with PBS buffer containing 0.05% Tween-20 (PBST), the magnetic beads were resuspended in PBS and added to 2 mL of fresh TG1 bacterial culture and placed in a shaking table at 37° C. and 100 rpm for 45 minutes to infect. After infection, 10 μL M13 helper phage was added to re amplify the phage for the next round of panning as an enriched library.

Four rounds of panning were carried out for random mutation library and 5, 4, 2, and 1 μg of hCD 16A antigen were used for each round respectively, number of PBST washing step was also increased gradually for each round of panning. At the end of 4^{th} round of panning, single colonies were randomly picked from the eluted phage infected TG1 cells, monoclonal ELISA was performed to identify CD16A specific binders. In brief, 96-well ELISA plates were coated with hCD16A protein and blocked with PBS buffer containing 2% BSA at 4° C. overnight, 100 μL of TG1 culture was then added to the plate and incubated at 37° C. for 30 min. After the plate was washed twice with PBS buffer containing 0.05% Tween 20, VH binding was detected by HRP-conjugated anti-Flag antibody. Top binders were picked and the corresponding phagemids in TG1 cell were isolated and subjected to DNA sequencing to identify enriched VHs. The enriched VHs were designated as CD16-VH-RM72, CD16-VH-RM73, CD16-VH-RM75, CD16-VH-RM76, CD16-VH-RM77, CD16-VH-RM80, CD16-VH-RM81, and CD16-VH-RM82. Amino acid sequences were listed for the enriched VHs in the Sequence section.

### Example 2: Protein Production of VH in E. coli and Characterization of Binding to CD16 158F/V Protein by ELISA and Biolayer Interferometry

Selected VHs from phage panning were subjected to further verification using purified antibodies. This example illustrates protein production of CD16A VH in E Coli and characterization of binding to hCD16A protein by ELISA and biolayer interferometry (BLI).

Selected VHs plasmids were transformed into HB2151 competent cells. A single colony from an overnight-grown ampicillin plate was inoculated into SB bacterial culture medium, and bacteria culture was cooled down to 30-degree before adding IPTG to induce protein expression. Bacteria cultures were harvested and VHs were purified using Ni-NTA column following manufacturer's instructions. The purified antibody was subjected to in vitro binding characterizations by ELISA. In brief, human CD16 158V and 158F protein (Seq ID NO: 2 and Seq ID NO: 11) were coated on the ELISA plate at 4° C. overnight, after washing with PBS containing 0.05% Tween20 (PBST), a serially diluted VHs were added to coated ELISA plate and incubated at room temperature for 1 hour, and the binding ability of the VHs to CD16A was detected with an anti-FLAG tag antibody.

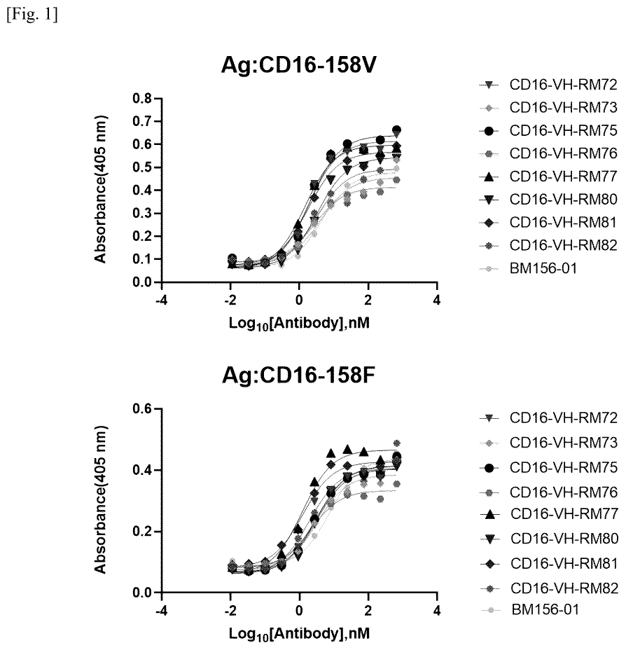

In experiments performed essentially as described above, comparing to BM156-01, several clones with lower binding EC₅₀ such as CD16-VH-RM72, CD16-VH-RM77 and CD16-VH-RM81 were obtained from panning. The ELISA results were shown in FIG. 1 and EC₅₀ were shown in Table 2.

TABLE 2
Binding EC₅₀ of different VHs to CD16A
158V and CD16A 158F determined by ELISA

| | CD16A-158V | CD16A-158F |
|---|---|---|
| Antibody | EC₅₀ (nM) | EC₅₀ (nM) |
| BM156-01 | 4.568 | 7.658 |
| CD16-VH-RM72 | 1.86 | 1.893 |
| CD16-VH-RM73 | 4.197 | 3.288 |
| CD16-VH-RM75 | 2.055 | 3.041 |
| CD16-VH-RM76 | 2.715 | 2.199 |
| CD16-VH-RM77 | 1.423 | 1.212 |
| CD16-VH-RM80 | 3.323 | 3.034 |
| CD16-VH-RM81 | 1.797 | 1.247 |
| CD16-VH-RM82 | 3.201 | 3.287 |

The binding of CD16A VHs to CD16A protein were also characterized by BLI. Biotinylated CD16A-158V or 158F were immobilized onto avidin coated probe, as recommended by the manufacturer (Octet). CD16A VHs were 1:2 serially diluted from 500 nM to 31.3 nM. Equilibrium dissociation constants (K_{D}) for monovalent receptor binding were determined by fitting 1:1 Langmuir model to the data.

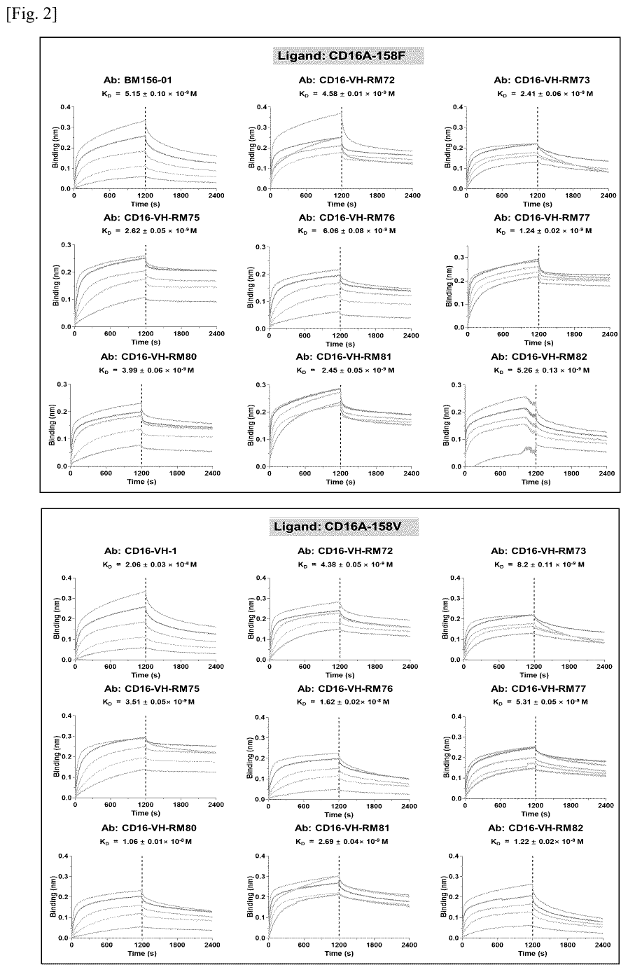

In experiments performed essentially as described above, Comparing to BM156-01, the binding affinity of CD16-VH-RM72, CD16-VH-RM75, CD16-VH-RM77 and CD16-VH-RM81 to CD16A-158F/V were all improved. The results were shown in FIG. 2 and table 3.

TABLE 3
Binding affinity of different single domain antibodies
to CD16A 158V and CD16A 158 determined by BLI.

| | CD16A-158V | CD16A-158F |
|---|---|---|
| Antibody | EC₅₀ (nM) | EC₅₀ (nM) |
| BM156-01 | 20.6 ± 0.3 | 5.15 ± 0.10 |
| CD16-VH-RM72 | 4.38 ± 0.05 | 4.58 ± 0.01 |
| CD16-VH-RM73 | 8.2 ± 0.11 | 2.41 ± 0.06 |
| CD16-VH-RM75 | 3.51 ± 0.05 | 2.62 ± 0.05 |
| CD16-VH-RM76 | 16.2 ± 0.2 | 6.06 ± 0.08 |
| CD16-VH-RM77 | 5.31 ± 0.05 | 1.24 ± 0.02 |
| CD16-VH-RM80 | 10.6 ± 0.1 | 3.99 ± 0.6 |
| CD16-VH-RM81 | 2.69 ± 0.04 | 2.45 ± 0.05 |
| CD16-VH-RM82 | 12.2 ± 0.2 | 5.26 ± 0.13 |

### Example 3: Design Bispecifics with Different Format and N297A and LALA Mutations

This example shows construction and engineering of bispecific with different format.

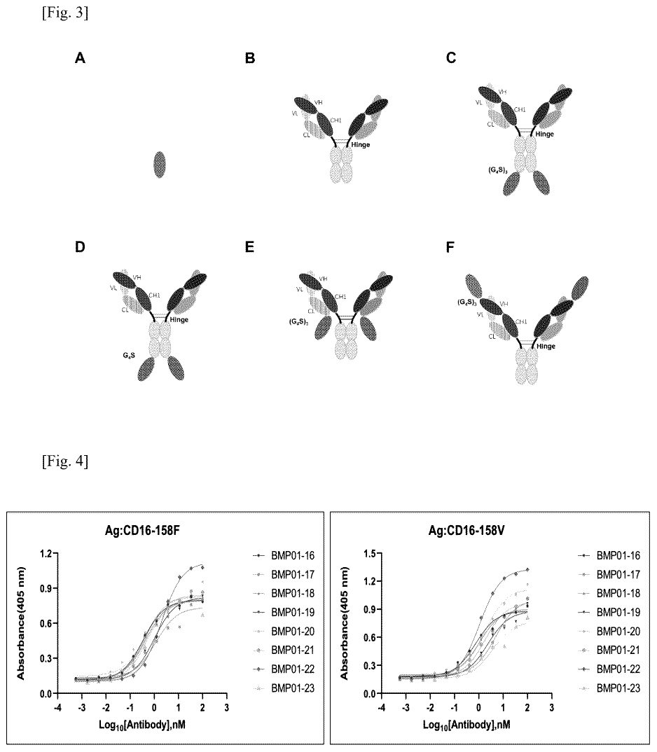

BM130-93 (Trastuzumab Seq ID NO: 12 and Seq ID NO: 13) was chosen as a building block together with selected CD16A VHs to construct bispecific for its success treating HER2+ breast and gastric cancer in clinic. CD16A VH can be fused to C-terminal of BM130-93 heavy chain as morrison body with (G4S) 3 linker (Seq ID NO: 14 and Seq ID NO: 13), or (G4S) 4 linker (Seq ID NO: 15 and Seq ID NO: 13). CD16A VH can also be fused to the C terminus of light chain (Seq ID NO: 12 and Seq ID NO: 16), or be placed at the N-terminal of heavy chain (Seq ID NO: 17 and Seq ID NO: 13). These different formats are illustrated in FIG. 3.

Bispecific antibodies BMP01-16, to BMP01-23 exemplified in the examples were constructed by fusing CD16A VH to C-terminal of BM130-93 heavy chain with (G4S) 3 linker, the light chain of BM130-93 was not modified.

### Example 4: CHO Production and Binding Characterization of Bispecific to CD16A by ELISA and BLI

This example demonstrates CHO production and binding characterization of bispecific to CD16A protein by ELISA and BLI.

Different heavy chain together with corresponding light chain were transiently co-expressed in the ExpiCHO cell line to generate bispecific antibodies. The protein from the ExpiCHO supernatant was purified using Capturem™ Protein A Miniprep Columns (Takarabio, #635717) following manufacturer's instructions. The purified antibody was subjected to in vitro binding and functional characterizations.

The human CD16 158V and 158F protein (Seq ID NO: 2 and Seq ID NO: 11) were coated on the ELISA plate 4° C. overnight, after washing with PBS containing 0.05% Tween20 (PBST). Serially diluted bispecific antibody BMP01 were added to coated ELISA plate and incubated at room temperature for 1 hour, and the binding ability of bispecific antibody to CD16 was detected with an anti-human Fc tag antibody. The ELISA results showed the binding of BMP01-16 to BMP01-23 bispecific antibodies to CD16A 158V and 158F remain. Detailed results were shown in FIG. 4.

The binding affinity of bispecific antibodies to CD16A protein were characterized by BLI. Biotinylated CD16A-158V or 158F were immobilized onto avidin coated probe, as recommended by the manufacturer (Octet). Bispecific antibodies and BM130-93 (Trastuzumab) were 1:2 serially diluted from 500 nM to 31.3 nM. Equilibrium dissociation constants (K_{D}) for monovalent receptor binding were determined by fitting 1:1 Langmuir model to the data.

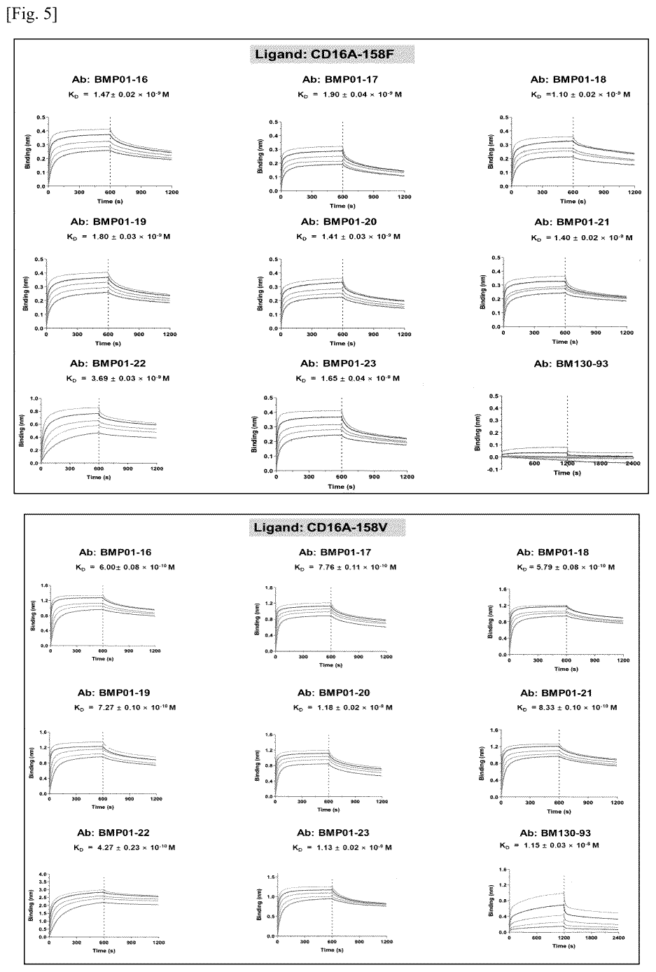

In experiments performed essentially as described above, bispecific antibodies BMP01-16 to 19, BMP01-21, 22 all bonded to CD16A-158V with affinity below nanomolar. Comparing to BM130-93 (Trastuzumab) which had no apparent binding to CD16A 158F variant, BMP01-16 to 23 all showed strong binding with affinity around nanomolar. The detailed results were shown in FIG. 5 and Table 4.

TABLE 4
Binding affinity of different bispecific antibodies to CD16A 158V and CD16A 158F

| Antigen | Antibody | K_{D}(M) | Kₒₙ(Ms⁻¹) | K_{off}(s⁻¹) |
|---|---|---|---|---|
| CD16-158V- | BMP01-16 | 6.00 ± 0.08 × 10⁻¹⁰ | 8.08 ± 0.09 × 10⁵ | 4.84 ± 0.04 × 10⁻⁴ |
| Biotin | BMP01-17 | 7.76 ± 0.11 × 10⁻¹⁰ | 9.31 ± 0.12 × 10⁵ | 7.23 ± 0.05 × 10⁻⁴ |
| | BMP01-18 | 5.79 ± 0.08 × 10⁻¹⁰ | 7.57 ± 0.07 × 10⁵ | 4.38 ± 0.04 × 10⁻⁴ |
| | BMP01-19 | 7.27 ± 0.10 × 10⁻¹⁰ | 7.42 ± 0.08 × 10⁵ | 5.40 ± 0.04 × 10⁻⁴ |
| | BMP01-20 | 1.18 ± 0.02 × 10⁻⁹ | 7.40 ± 0.10 × 10⁵ | 8.70 ± 0.05 × 10⁻⁴ |
| | BMP01-21 | 8.33 ± 0.10 × 10⁻¹⁰ | 6.30 ± 0.06 × 10⁵ | 5.25 ± 0.04 × 10⁻⁴ |
| | BMP01-22 | 4.27 ± 0.23 × 10⁻¹⁰ | 1.91 ± 0.02 × 10⁵ | 8.14 ± 0.43 × 10⁻⁵ |
| | BMP01-23 | 1.13 ± 0.02 × 10⁻⁹ | 5.01 ± 0.06 × 10⁵ | 5.68 ± 0.06 × 10⁻⁴ |
| | BM130-93 | 1.15 ± 0.03 × 10⁻⁸ | 5.84 ± 0.12 × 10⁴ | 6.74 ± 0.06 × 10⁻⁴ |
| CD16-158F- | BMP01-16 | 1.47 ± 0.02 × 10⁻⁹ | 4.89 ± 0.07 × 10⁵ | 7.21 ± 0.06 × 10⁻⁴ |
| Biotin | BMP01-17 | 1.90 ± 0.04 × 10⁻⁹ | 7.19 ± 0.16 × 10⁵ | 1.36 ± 0.09 × 10⁻³ |
| | BMP01-18 | 1.10 ± 0.02 × 10⁻⁹ | 6.54 ± 0.12 × 10⁵ | 7.22 ± 0.08 × 10⁻⁴ |
| | BMP01-19 | 1.80 ± 0.03 × 10⁻⁹ | 4.35 ± 0.06 × 10⁵ | 7.81 ± 0.07 × 10⁻⁴ |
| | BMP01-20 | 1.41 ± 0.03 × 10⁻⁹ | 6.69 ± 0.12 × 10⁵ | 9.41 ± 0.08 × 10⁻⁴ |
| | BMP01-21 | 1.40 ± 0.02 × 10⁻⁹ | 6.33 ± 0.11 × 10⁵ | 6.77 ± 0.07 × 10⁻⁴ |
| | BMP01-22 | 3.69 ± 0.06 × 10⁻⁹ | 1.20 ± 0.01 × 10⁵ | 4.44 ± 0.05 × 10⁻⁵ |
| | BMP01-23 | 1.65 ± 0.04 × 10⁻⁹ | 5.61 ± 0.11 × 10⁵ | 9.28 ± 0.08 × 10⁻⁴ |
| | BM130-93 | NO | / | / |

### Example 5: Dual Binding of BMP01 to Two Targets Simultaneously

This example demonstrates that BMP01 antibodies can bind to dual targets simultaneously using a sandwich ELISA assay.

50 μL of PBS solution containing Human HER2/ErbB2 Protein (His Tag, 1 μg/mL) (Seq ID NO: 32) or BSA (1 μg/mL) were added to a 96-well microplate, and place the antigen solution overnight at 4° C. to prepare HER2 antigen coated ELISA plate. Discard the coating solution the next day, wash the plate three times with 300 μl/well PBST solution containing 0.05% Tween20 (500 μl Tween20+1000 ml PBS), add 300 μl/well PBS containing 4% skimmed milk powder, and place at 37° C. for 1 hour to block non-specific binding. Control samples and the sample to be tested was pre-diluted to 15 μg/ml (the first point concentration) with PBS, and then serially diluted 2.5 times, with a total of 8 concentration points. After blocking, the plate was washed 3 times with PBST, 50 μl/well of the diluted control sample and test sample were added, and incubated at 300 rpm at 37° C. for 1.5 h. Wash the plate 3 times with PBST solution, add 1 μg/ml biotin-labeled 158V CD16A-His 50 μl/well, 300 rpm, 37° C., 1.5 h. Wash the plate three times with PBST solution again, add 1:5000 diluted detection antibody SA-HRP 50 μl/well, and incubate at 300 rpm at 37° C. for 1.5 h. Wash the plate 6 times with PBST solution, add 50 μl/well of chromogenic solution equilibrated to room temperature beforehand, and develop color at room temperature for 15 minutes in the dark. Add stop solution 1M HCl, 25 μl/well. A microplate reader was used to detect the OD value at 450 nm.

The OD values were subjected to 4-parameter fitting to calculate EC₅₀ with Concentration as the abscissa and Mean Values as the ordinate.

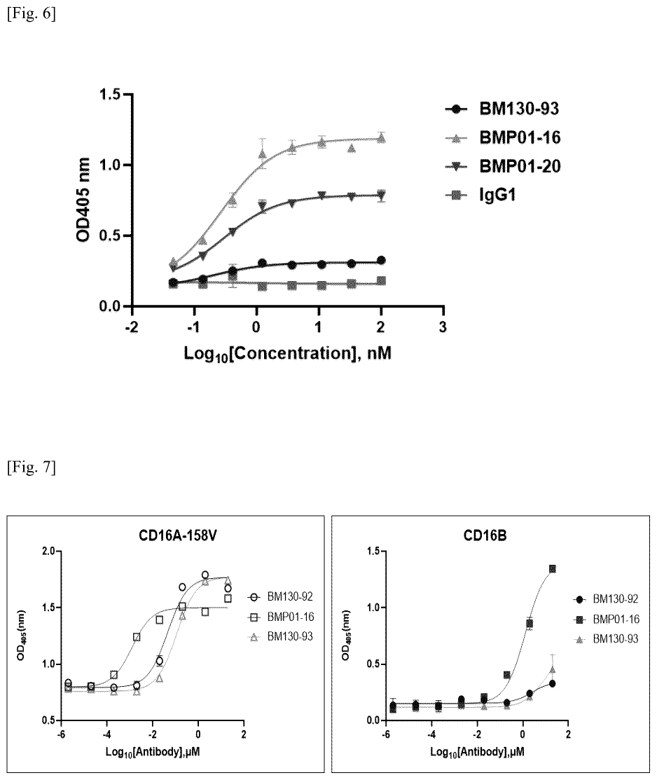

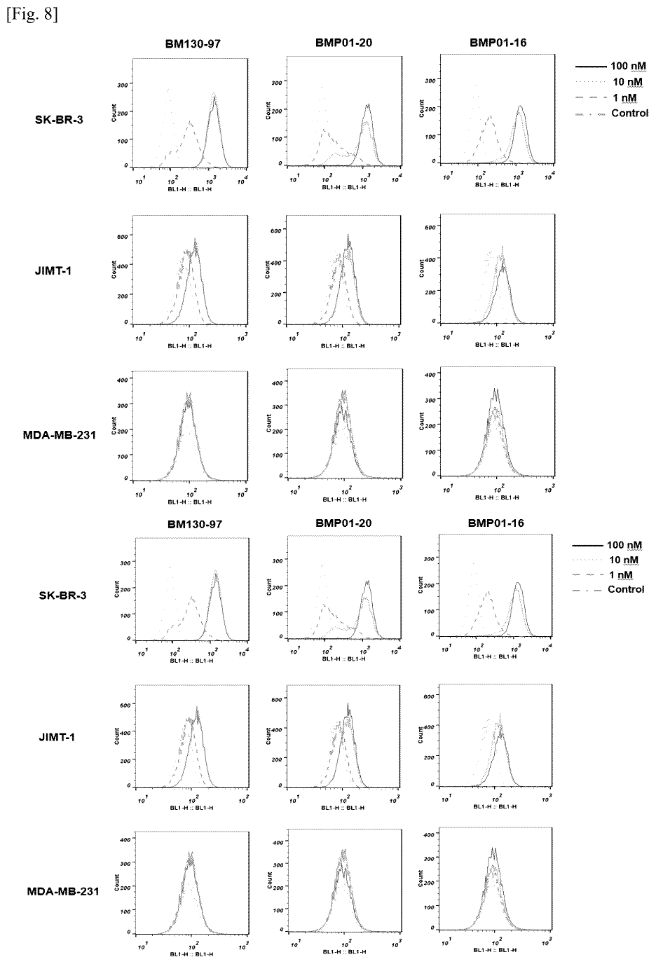

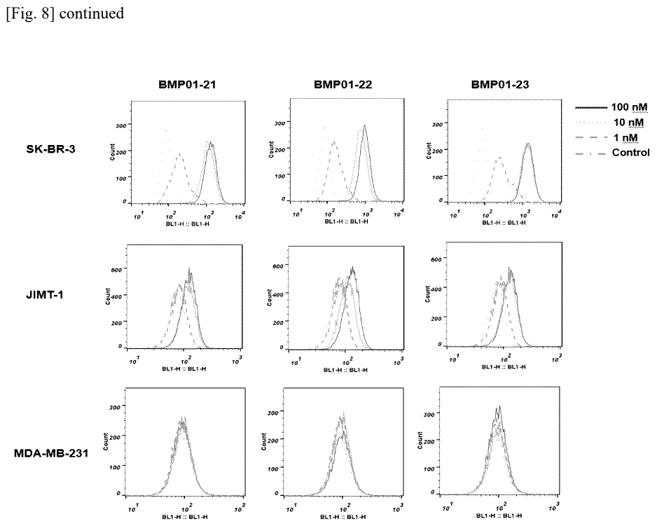

In experiments performed essentially as described above, only BMP01-16 and BMP01-20 were able to bind to 2 targets simultaneously with EC₅₀ at 0.2642 nM for BMP01-16 and 0.2947 nM for BMP01-20, while BMP130-93 or isotype control not. Detailed result was shown in FIG. 6.

### Example 6: Cross-Binding with CD16A from Different Species

The binding of bispecific antibodies to CD16 protein from different species including human, monkey (Seq ID NO: 33), mouse (Seq ID NO: 34) and rat (SEQ ID NO: 35) were characterized by BLI. Biotinylated CD16A 158V or 158F were immobilized onto avidin coated probe, as recommended by the manufacturer (Octet). BMP01-16 were 1:2 serially diluted from 200 nM while Trastuzumab were 1:2 serially diluted from 140 nM. Equilibrium dissociation constants (K_{D}) for monovalent receptor binding were determined by fitting 1:1 Langmuir model to the data.

In experiments performed essentially as described above, bispecific antibodies BMP01-16 bonded to human and monkey CD16A with high affinity at 0.11 nM and 3.31 nM respectively. BMP01-16 showed much weak binding to mouse or rat CD16 protein with affinity at 0.645 μM and 0.0958 μM. Trastuzumab however only showed weak binding to CD16 from all species. The detailed results were shown in Table 5.

TABLE 5
Binding affinity of BMP01-16 and Trastuzumab
to human, monkey, mouse and rat CD16 protein

| Antibody | Antigen | koff(1/s) | kon(1/Ms) | K_{D} (M) | FullR2 |
|---|---|---|---|---|---|
| BMP01-16 | human | 3.29E−05 | 2.99E+05 | 1.10E−10 | 0.996 |
| Trastuzumab | CD16A(158V) | 0.0063 | 1.16E+05 | 5.44E−08 | 0.988 |
| BMP01-16 | Cynomolgus Fc | 2.00E−03 | 6.05E+05 | 3.31E−09 | 9.86E−01 |
| Trastuzumab | gamma RIII/CD16 | 8.86E−03 | 2.47E+05 | 3.59E−08 | 9.84E−01 |
| BMP01-16 | Mouse Fc | 0.0374 | 5.80E+04 | 6.45E−07 | 9.73E−01 |
| Trastuzumab | gamma RIII/CD16 | 8.93E−03 | 3.43E+05 | 2.60E−08 | 9.55E−01 |
| BMP01-16 | CD16A Protein, | 5.60E−03 | 5.84E+04 | 9.58E−08 | 9.75E−01 |
| Trastuzumab | Rat | 3.65E−03 | 3.56E+04 | 1.03E−07 | 9.70E−01 |

### Example 7: CD16B Binding

The human CD16A and CD16B (Seq ID NO: 36) proteins were coated on ELISA plate at 4° C. overnight. After washing with PBS containing 0.05% Tween20 (PBST), serially diluted BMP01-16 and BM130-92 (Margetuximab, Seq ID NO: 37 and Seq ID NO: 38) and BM130-93 (Trastuzumab) antibody were added to coated ELISA plate and incubated at room temperature for 1 hour, and the binding ability of bispecific and control antibody to CD16 was detected with an anti-human Fc tag antibody.

In experiments performed essentially as described above, binding of BMP01-16 to CD16A-158V and CD16B were 1.255 nM and 1.296 μM respectively. This result suggested that BMP01-16 had much stronger binding to CD16A-158V than CD16B protein with EC₅₀>1000 fold. In contrast, BM130-92 (Margetuximab) and BMP130-93 (Trastuzumab) bonded to CD16A-158V with EC₅₀ at 45.09 nM and 110.9 nM respectively while bindings to CD16B were negligible. Details results see FIG. 7 and Table 6.

TABLE 6
EC₅₀ for Binding of BMP01-16, BM130-92 and BM130-
93 to human CD16A-158V and CD16B on ELISA

| EC₅₀ (μM) | Margetuximab | BMP01-16 | Herceptin |
|---|---|---|---|
| CD16A-158V | 0.04509 | 0.001255 | 0.1109 |
| CD16B | NA | 1.296 | NA |

### Example 8: Evaluation of Cell Surface HER2 Binding of Bispecific Antibody

This example demonstrates binding of BMP01 antibodies to cell surface HER2 evaluated by FACS analysis.

5×10⁵ tumor cell with different HER2 expression level, for example SK-BR-3 cells (high expression), JIMT-1 (medium expression) or MDA-MB-231 (low expression) were blocked with human TruStain FcX™ (Biolegend, 422302) for 10 minutes at room temperature before incubation with various concentration of antibody variants (100 nM, 10 nM, 1 nM) for 30 min at 4° C., IgG1 was used as isotype control. After incubation, cells were washed with 3% BSA in PBS. Cell-bound antibody levels were detected by APC-conjugated, AffiniPure F(ab′)₂ Fragment Goat Anti-Human IgG (Jackson ImmunoResearch Laboratories). The geometric mean fluorescent intensity was measured on Attune cell analyzer (Invitrogen). Data were analyzed using Flowjo Software (Tree star Inc).

In experiments performed essentially as described above, results showed that BM130-93 and all BMP01 antibody variants bonded to cell surface HER2 to a similar extent. The higher HER2 receptor expression on cell surface, the more cell bound BM130-93 and BMP01 antibodies could be detected.

### Example 9: ADCC Reporter Assay

This example demonstrates evaluation of ADCC biological activity of BMP01 antibody by luciferase reporter assay using Jurkat/NFAT-luc-CD16A transgenic cell line as effector cells and HER2 or 5T4 expressing tumor cell line as target cells.

The day before the ADCC experiment, HER2+ or 5T4+ target cells were inoculated onto 96 plates at a density of 1×10⁴ per well, 100 μL/well, and cultured overnight at 37° C. in a 5% CO2 incubator. On the day of the experiment, the antibody to be tested was diluted 5 times in RPMI-1640 medium containing 10% heat-inactivated FBS. 50 μL diluted antibody was added to each well containing target cells. BM130-93 (Trastuzumab) or m603 (anti-5T4 antibody, Seq ID NO: 29 and Seq ID NO: 30) was used as positive control. Then 1×10⁴ Jurkat-NFAT-Luc2-CD16A-158V or Jurkat-NFAT-Luc2-CD16A-158F effector cells (50 μL/well) were added to the wells, and cultured further in a 5% CO₂ incubator at 37° C. for 6 hours. At the endo of incubation, 100 μl of Steady-Glo Luciferase Assay System reaction substrate was added to each well, pipette repeatedly to lyse the cells, leave at room temperature for 5-30 min, then transfer the lysate to a 96-well white plate to avoid the generation of air bubbles, and use a microplate reader to detect the fluorescence value.

The ADCC Luciferase induction fold is calculated by the fold change of the relative light unit (RLU) value of the test well and the control well (simulated buffer reaction with target cells and effector cells): Fold change=(RLU test drug-RLU culture medium control)/(RLU NC-RLU culture medium control), using GraphPad Prism software for statistical analysis and graphing.

Culture medium control: only add culture medium

NC control: target cells+effector cells without samples to be tested

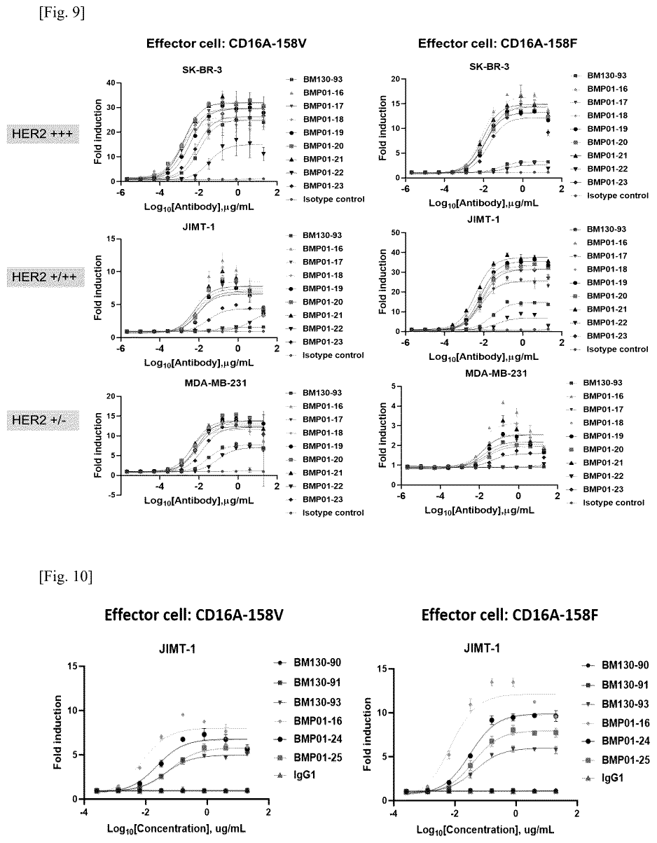

In experiments performed essentially as described above, all antibodies treatments resulted in induction of ADCC in a dose and target cell dependent manner. BMP01 demonstrated superior ADCC activity comparing to BM130-93 (Trastuzumab) with regards to EC₅₀ (Table 7) and maximum fold induction. Detailed results were shown in FIG. 9.

TABLE 7
EC₅₀ of luciferase activity of Jurkat T-CD16A-158F
or 158V cells after incubation with antibodies

| | | SK-BR-3 | JIMT-1 | MDA-MB-231 |
|---|---|---|---|---|
| Effector cell | Antibody | EC₅₀ (μg/mL) | EC₅₀ (μg/mL) | EC₅₀ (μg/mL) |
| CD16-158V | BMP01-16 | 0.001 | 0.005 | 0.009 |
| | BMP01-17 | 0.002 | 0.009 | 0.015 |
| | BMP01-18 | 0.001 | 0.007 | 0.010 |
| | BMP01-19 | 0.004 | 0.009 | 0.013 |
| | BMP01-20 | 0.002 | 0.008 | 0.010 |
| | BMP01-21 | 0.002 | 0.006 | 0.009 |
| | BMP01-22 | 0.053 | 0.106 | 0.788 |
| | BMP01-23 | 0.007 | 0.018 | 0.037 |
| | BM130-93 | 0.010 | 0.032 | 0.068 |
| CD16-158F | BMP01-16 | 0.010 | 0.005 | 0.005 |
| | BMP01-17 | 0.016 | 0.008 | 0.008 |
| | BMP01-18 | 0.014 | 0.006 | 0.006 |
| | BMP01-19 | 0.013 | 0.008 | 0.008 |
| | BMP01-20 | 0.013 | 0.009 | 0.009 |
| | BMP01-21 | 0.011 | 0.004 | 0.004 |
| | BMP01-22 | 0.267 | 0.087 | 0.087 |
| | BMP01-23 | 0.023 | 0.009 | 0.009 |
| | BM130-93 | 0.076 | 0.027 | 0.27 |

Once L234 and L235 in Fc portion of Trastuzumab or N297 were mutated to alanine to generate variants BM130-90 and BM130-91, both variants lost their ADCC activities completely while BM130-93 retained its activity. However, BMP01-16 LALA variant BMP01-24 and N297A variant BMP01-25 were still active in the same assay, although slightly less. This result suggested CD16A VH made significant contribution to superior NK stimulation activity of BMP01 antibodies. Detailed results were shown in FIG. 10.

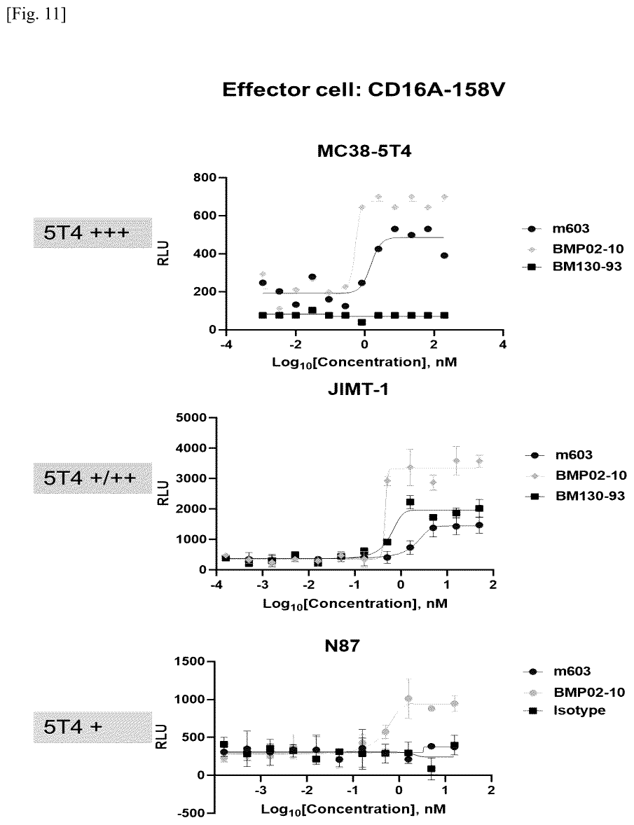

In experiments based on experiments performed as described above, BMP02-10 (Seq ID NO: 30 and Seq ID NO: 31) treatment enhanced ADCC activity to various extent, depending on the cell type and 5T4 expression level. Comparing to control 5T4 antibody m603, BMP02-10 exhibited superior ADCC activity in terms of EC₅₀ and maximum fold induction, for example, EC₅₀ for BMP02-10 and m603 were 2.8 nM and 95 nM respectively in JIMT-1 assay. The detailed results were shown in FIG. 11 and Table 8.

TABLE 8
EC₅₀ of luciferase activity of Jurkat T-CD16A-
158V cells after incubation with antibodies

| | | MC38-5T4 | JIMT-1 | N87 |
|---|---|---|---|---|
| Effector cell | Antibody | EC₅₀ (nM) | EC₅₀ (nM) | EC₅₀ (nM) |
| CD16-158V | BMP02-10 | 0.53 | 2.8 | 2.7 |
| | m603 | 1.5 | 95 | ND |

### Example 10: NK Cell Killing Assay

This example demonstrates enhancement of NK cell killing activity of BMP01 antibody by lactate dehydrogenase release assay using PBMC as effector cells and HER2 or 5T4-expressing tumor cell lines as target cells.

One day before NK killing experiment, 2×10⁴/100 μl target cells were seeded in a 96 plate and incubated overnight at 37° C. in a 5% CO₂ incubator. On the day of the experiment, the antibody to be tested was serially diluted 5 times with RPMI-1640 medium. The target cell culture medium was discarded, 50 μL of fresh medium was added, and 50 μL diluted antibody were added to each well containing target cells. Then 5×10⁵ PBMC cells isolated from fresh blood were added to each well at 100 μL/well. Set up the following three groups of controls at the same time. (1) Target cell spontaneous release group: 50 μL target cells and 150 μL test medium without test molecules; (2) Antibody-independent cytotoxicity group: 50 μL target cells, 100 μL PBMC cells and 50 μL medium without test molecules; and (3) Target cell maximum release group: 50 μL target cells and 120 μL medium, 30 μL/well lysis solution was added at the end of the experiment to determine the maximum LDH release. Incubate further for 4-6 hours in a 5% CO₂ incubator at 37° C.

After incubation, 50 μL/well of LDH-containing medium supernatant was transferred to a 96-well transparent flat bottom plate, 50 μL of CytoTox 96® reagent was added to the supernatant and incubated at room temperature for 30 min in the dark, then 50 μL of stop solution was added and placed in Measure the absorbance at 490 nm or 492 nm within 1 hour.

The cell killing ratio was calculated by the following formula. Statistical analysis and graphing of data were carried out with GraphPad Prism software.

Cell killing %=(sample antibody sample OD-antibody-independent cytotoxicity group OD)/(target cell maximum release group OD-target cell spontaneous release group OD)*100

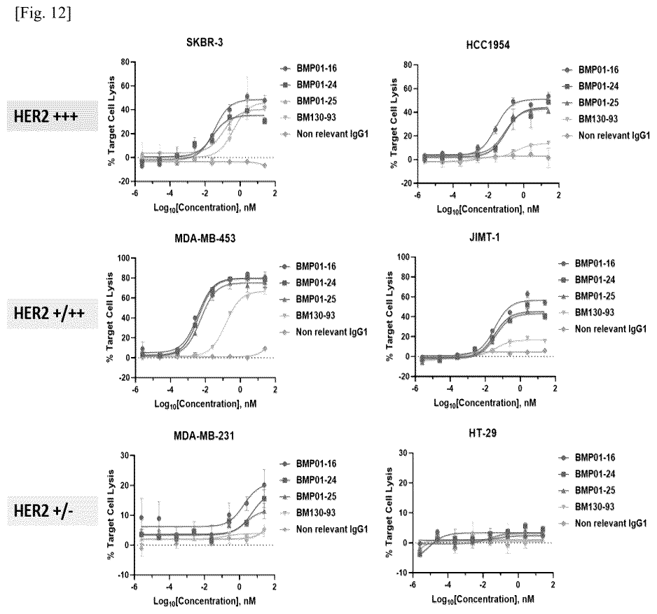

In experiments performed essentially as described above, BMP01 treatments result in various degree of induction of cell killing depending on cell type and HER2 expression levels. In all cases except HT-29, BMP01-16 demonstrated superior enhancement of NK killing activity compared to BM130-93 (Trastuzumab) with regards to EC₅₀ and maximum fold induction. In consistent with previous luciferase reporter assay, BMP01-16 LALA variant BMP01-24 and N297A variant BMP01-25 still retain stimulation of NK cell killing activity, although less than BMP01-16 in some cases. Detailed results shown in FIG. 12.

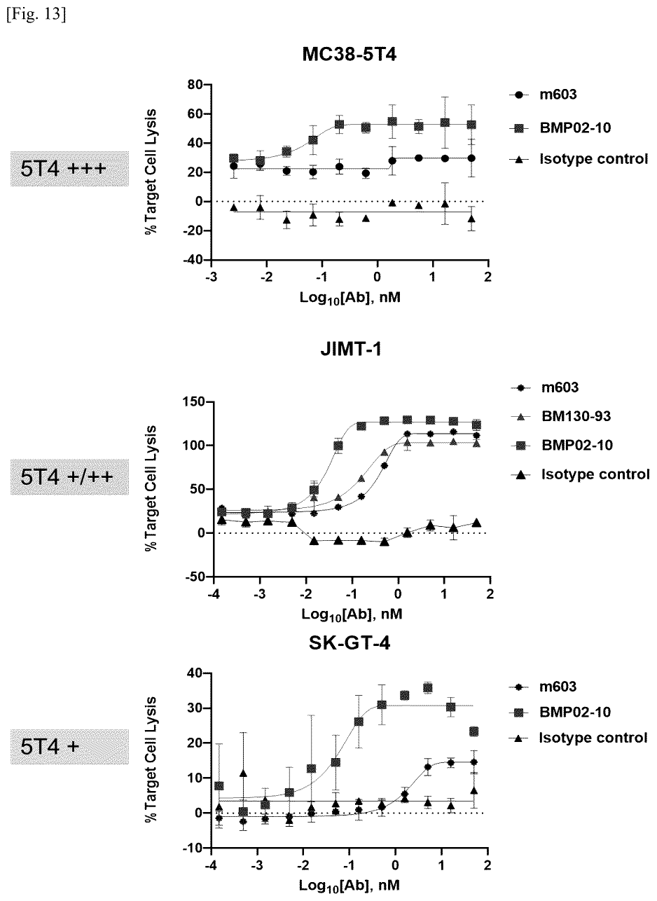

In experiments based on experiments performed as described above, BMP02-10 treatment enhanced NK cell killing towards target cells to various extent, depending on the cell type and 5T4 expression level. Comparing to control 5T4 antibody m603, BMP02-10 exhibited superior cell killing activity in terms of EC₅₀ and maximum fold induction. The detailed results were shown in FIG. 13.

### Example 11: In Vitro IFN-γ Release Assay

This example demonstrates innate immune cell stimulation activity of BMP01 evaluated by IFN-γ release assay using PBMC as effector cells and HER2-expressing tumor cell lines as target cells.

One day before IFN-γ release experiment, 2×10⁴/100 μl target cells were seeded into a 96 well plate and incubated overnight at 37° C. in a 5% CO₂ incubator. On the day of the experiment, the antibody to be tested was serially diluted 5 times with RPMI-1640 medium. The target cell culture medium was discarded, 50 μL of fresh medium was added, and 50 μL diluted antibodies were added to each well containing target cells. Then 5×10⁵ PBMC cells isolated from fresh blood were added to each well at 100 μL/well, incubated further for 72 hours in a 5% CO₂ incubator at 37° C. After incubation, 50 μL/well of culture supernatant were transferred to a 96-well transparent flat bottom plate, The concentrations of IFN-γ were determined by Human IFN-γ Quantikine ELISA Kit using assay conditions recommended by manufacture (R & D systems).

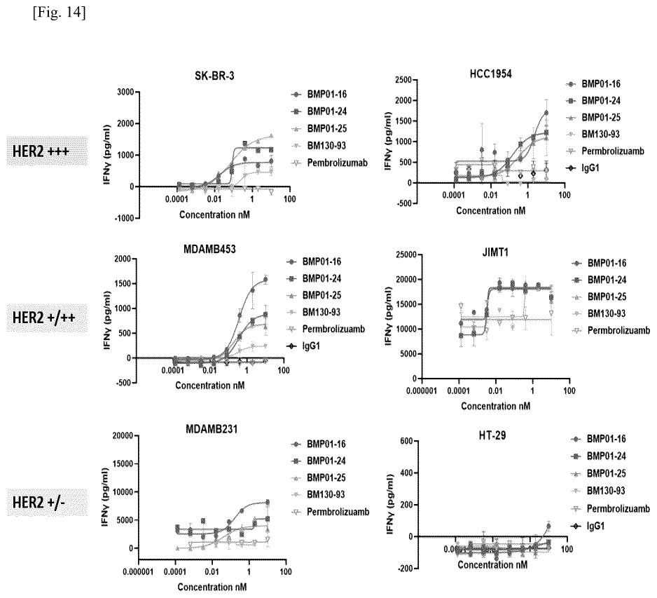

In experiments performed essentially as described above, BMP01-16 treatments resulted in various degree of IFN-γ induction depending on cell type and HER2 expression levels. In all cases except HT-29, BMP01-16 induced more IFN-γ production than BMP130-93, indicative of more NK cell activation induced by BMP01 bispecifics. In this assay BMP01-16 LALA variant BMP01-24 and N297A variant BMP01-25 exhibited mixed degree of enhancement comparing to BMP01-16. Detailed results were shown in FIG. 14.

### Example 12: In Vivo Efficacy of BMP01-16 in JIMT-1/PBMC Comix Mouse Model

This example demonstrates the in vivo efficacy testing of BMP01-16 antibody using the JIMT-1/PBMC co-mixed mouse model.

On day 0 fresh human PBMC cells (5×10⁶ cells) and JIMT-1 human breast cancer cells (1×10⁶) were premixed at an E/T ratio of 5:1 and implanted subcutaneously into 6-8 week-old female NOD/SCID mice (Jiangsu Jicui Yaokang Biotechnology Co., Ltd.). The engrafted mice were randomly divided into 8 groups with 5 mice in each group. Starting from day 0 mice were then treated with BMP130-93 or BMP01-16 intraperitoneally at three different dose levels (0.5, 1.5, 5 mg/kg for BMP130-93 and 0.6, 1.8, 6 mg/kg for BMP01-16) twice a week for 4 weeks. In addition, two control groups were set up, including the JIMT-1 cancer cell group and the vehicle group. Tumor volumes were recorded twice a week from the day of tumor cell implantation. Data analysis and graphing were performed using GraphPad Prism software.

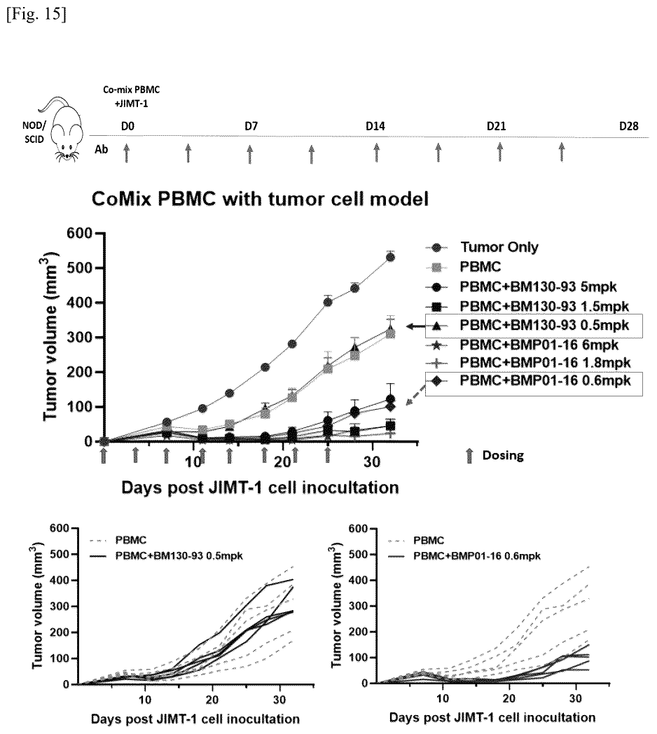

In experiments performed essentially as described above, there were significant tumor growth inhibition with medium and high doses of BMP130-93 (Trastuzumab) and BMP01-16 by day 32, the time point when the study was terminated. In contrast, low doses of BM130-93 (Trastuzumab) at 0.5 mg/kg had no tumor suppressive effect, but low doses of BMP01-16 at 0.6 mg/kg could significantly inhibit tumor growth, indicating better anti-tumor activity of BMP01-16 in this experimental setting. Detailed results were shown in FIG. 15.

### Example 13: In Vivo Efficacy of BMP01-16 in huHSC-NCG-hIL15/HCC1954 Model

This example demonstrates the in vivo efficacy testing of BMP01-16 antibody in human breast cancer HCC1954/huHSC-NCG-hIL15 mouse subcutaneous transplantation model.

NCG-IL15 mice were purchased from Jiangsu Jicui Yaokang Biotechnology Co., Ltd. Human CD34+ hematopoietic stem cells were transplanted into the tail vein of irradiated NCG-hIL15 mice to construct huHSC-NCG-hIL 15 human immune reconstitution mice. After 4-6 weeks, hCD45+ cells can be detected in peripheral blood accounting for 20% or more of living cells. The HCC1954 tumor cell line was purchased from Nanjing Kebai Biotechnology Co., Ltd. HCC1954 cells were cultured in RPMI-1640 medium containing 10% heat-inactivated FBS, and the tumor cells were collected when the required cell number was reached. For total 30 mice, 0.2 ml 5.0×10⁶ HCC1954 cells+50% Matrigel gel (PBS:Matrigel at 1:1 volume ratio) were injected subcutaneously into the right forelimb of each mouse for tumor engraftment.

Before grouping, the level of immune reconstitution in the peripheral blood of the mice was analyzed by flow cytometry, and the biomarker used were: hCD45, hCD3, hCD19, hCD4, hCD8, hCD56, hCD16. When the average tumor volume reached about 100-120 mm³, 18 tumor-bearing mice were randomly divided into 3 groups (6 mice per group) according to the tumor volume, body weight and immune reconstitution level of the mice, and the tumor volume CV within group is ≤⅓. The day of grouping was defined as PG-DO, and the test antibody was given on the day of random grouping. The doses were 0.5 mg/kg BM130-93 (Trastuzumab) and 0.6 mg/kg BMP01-16, respectively. Both antibodies were intraperitoneally administered twice a week in the following 5 weeks. Body weight and tumor volume were measured twice a week. Tumor growth inhibition (TGI) was calculated as follows: TG1%=(1-T/C)×100%, T and C were the average TVs of the treatment group and the control group at the end of the experiment. Data analysis was performed using GraphPad Prism software, and the results were shown as the mean±S.E.M of tumor volume.

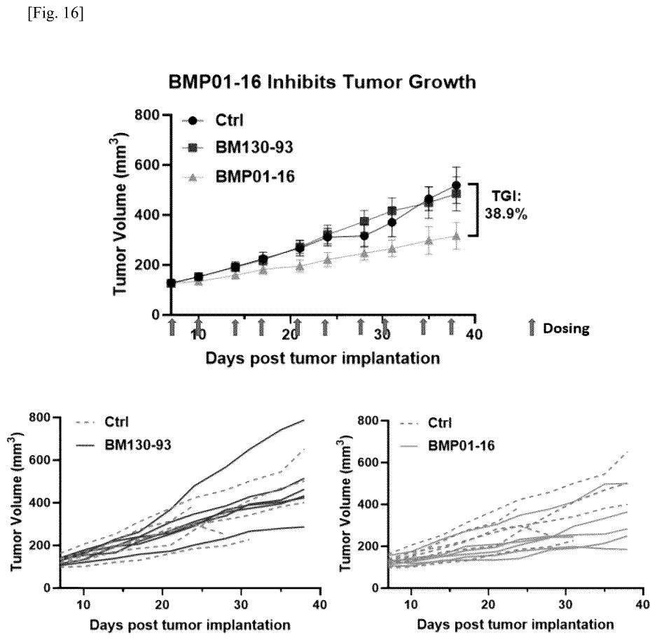

In experiments performed essentially as described above, treatments started when tumor reached about 100-120 mm³ in size. 0.6 mg/kg BMP01-16 had significant tumor growth inhibition with TG1 at 38.9% on day 38 after grouping, the time point at which the study was terminated. On the contrary, 0.5 mg/kg BM130-93 had no tumor inhibitory effect in this model. Detailed results were shown in FIG. 16.

1. A single domain antibody specifically binding to CD16A, wherein the single domain antibody has an amino acid sequence shown in SEQ ID NO: 1 comprising a substitution at a position selected from S54, G55, S56 and any combination thereof according to Kabat numbering system.

2. (canceled)

3. The single domain antibody according to claim 1, wherein the substitution is selected from a group consisting of: (i) S54N; (ii) S54D; (iii) S54T; (iv) G55V; (v) S56Q; (vi) S56T; (vii) S56D; (viii) S56E; (ix) S54N and S56Q; and (x) S54T and S56Q.

4. The single domain antibody according to claim 1, wherein the single domain antibody comprises:
(a) CDR1 having an amino acid sequence as shown in SEQ ID NO: 41 or a conservatively modified variant thereof, CDR3 having an amino acid sequence as shown in SEQ ID NO: 42 or a conservatively modified variant thereof, and CDR2 having an amino acid sequence selected from a group consisting of SEQ ID NO: 43, 44, 45, 46, 47, 48, 49, 50, 51, 52, and a conservatively modified variant each thereof, according to IMGT definition scheme;
(b) CDR1 having an amino acid sequence as shown in SEQ ID NO: 53 or a conservatively modified variant thereof, CDR3 having an amino acid sequence as shown in SEQ ID NO: 54 or a conservatively modified variant thereof, and CDR2 having an amino acid sequence selected from a group consisting of SEQ ID NO: 55, 56, 57, 58, 59, 60, 61, 62, 63, 64, and a conservatively modified variant each thereof, according to Kabat definition scheme;
(c) CDR1 having an amino acid sequence as shown in SEQ ID NO: 65 or a conservatively modified variant thereof, CDR3 having an amino acid sequence as shown in SEQ ID NO: 66 or a conservatively modified variant thereof, and CDR2 having an amino acid sequence selected from a group consisting of SEQ ID NO: 67, 68, 69, 70, 71, 72, 73, 74, 75, 76, and a conservatively modified variant each thereof, according to Chothia definition scheme; or
(d) CDR1 having an amino acid sequence as shown in SEQ ID NO: 77 or a conservatively modified variant thereof, CDR3 having an amino acid sequence as shown in SEQ ID NO: 78 or a conservatively modified variant thereof, and CDR2 having an amino acid sequence selected from a group consisting of SEQ ID NO: 79, 80, 81, 82, 83, 84, 85, 86, 87, 88, and a conservatively modified variant each thereof, according to Contact definition scheme;
with the proviso that the single domain antibody does not have an amino acid sequence shown in SEQ ID NO: 1.

5. The single domain antibody according to claim 4, wherein the single domain antibody comprises an amino acid sequence selected from a group consisting of SEQ ID NO: 3, 4, 5, 6, 7, 8, 9, 10, 39, 40, and an amino acid sequence having at least 80% identity to each thereof.

6. The single domain antibody according to claim 1, wherein the single domain antibody is a humanized antibody, a human antibody, a chimeric antibody, or a camelized antibody.

7. A heavy chain antibody comprising a single domain antibody according to claim 1, and an Fc portion linked to the single domain antibody.

8. A bispecific antibody comprising a single domain antibody according to claim 1.

9. The bispecific antibody according to claim 8, wherein the bispecific antibody comprises the single domain antibody binding specifically to CD16A, and a second binding domain specifically binding to a second target selected from a tumor associated antigen and a tumor specific antigen; optionally, the second target is selected from a group consisting of HER2, 5T4, PSMA, BCMA, FGFR, CD20, CD33, CD19, CD22, CD123, CD30, GPC-3, CEA, EGFR1, EGFR2, EGFR3, TGF-β, ROR1, PD-L1, Claudin18.2, EpCAM, GD2, MSLN, EGFR, MUC1, MUC2, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, MG7-Ag, NY-CO-1, RCAS 1, SDCCAG16, TAAL6 and TAG72.

10. (canceled)

11. The bispecific antibody according to any of claims 8 to 10 claim 9, wherein the single domain antibody is linked to a complete IgG antibody, optionally via a peptide linker; optionally, the peptide linker consists of glycine and serine residues; optionally, the single domain antibody is linked to the C terminus of a heavy chain, the C terminus of a light chain, or the N terminus of a heavy chain, of the complete IgG antibody.

12. (canceled)

13. (canceled)

14. The bispecific antibody according to claim 11, wherein the complete IgG antibody is an anti-HER2 antibody or an anti-5T4 antibody.

15. The bispecific antibody according to claim 14, wherein the anti-HER2 antibody comprises:
(a) a heavy chain having an amino acid sequence shown in SEQ ID NO: 12, and a light chain having an amino acid sequence shown in SEQ ID NO: 13;
(b) a heavy chain having an amino acid sequence shown in SEQ ID NO: 37, and a light chain having an amino acid sequence shown in SEQ ID NO: 38;
(c) a heavy chain having an amino acid sequence shown in SEQ ID NO: 25, and a light chain having an amino acid sequence shown in SEQ ID NO: 13; or
(d) a heavy chain having an amino acid sequence shown in SEQ ID NO: 26, and a light chain having an amino acid sequence shown in SEQ ID NO: 13.

16. The bispecific antibody according to claim 14, wherein the anti-5T4 antibody comprises a heavy chain having an amino acid sequence shown in SEQ ID NO: 29, and a light chain having an amino acid sequence shown in SEQ ID NO: 30.

17. The bispecific antibody according to claim 14, wherein the bispecific antibody comprises:
(a) a heavy chain having an amino acid sequence shown in SEQ ID NO: 14, and a light chain having an amino acid sequence shown in SEQ ID NO: 13;
(b) a heavy chain having an amino acid sequence shown in SEQ ID NO: 15, and a light chain having an amino acid sequence shown in SEQ ID NO: 13;
(c) a heavy chain having an amino acid sequence shown in SEQ ID NO: 17, and a light chain having an amino acid sequence shown in SEQ ID NO: 13;
(d) a heavy chain having an amino acid sequence shown in SEQ ID NO: 12, and a light chain having an amino acid sequence shown in SEQ ID NO: 16;
(e) a heavy chain having an amino acid sequence shown in SEQ ID NO: 18, and a light chain having an amino acid sequence shown in SEQ ID NO: 13;
(f) a heavy chain having an amino acid sequence shown in SEQ ID NO: 19, and a light chain having an amino acid sequence shown in SEQ ID NO: 13;
(g) a heavy chain having an amino acid sequence shown in SEQ ID NO: 20, and a light chain having an amino acid sequence shown in SEQ ID NO: 13;
(h) a heavy chain having an amino acid sequence shown in SEQ ID NO: 21, and a light chain having an amino acid sequence shown in SEQ ID NO: 13;
(i) a heavy chain having an amino acid sequence shown in SEQ ID NO: 22, and a light chain having an amino acid sequence shown in SEQ ID NO: 13;
(j) a heavy chain having an amino acid sequence shown in SEQ ID NO: 23, and a light chain having an amino acid sequence shown in SEQ ID NO: 13;
(k) a heavy chain having an amino acid sequence shown in SEQ ID NO: 24, and a light chain having an amino acid sequence shown in SEQ ID NO: 13;
(l) a heavy chain having an amino acid sequence shown in SEQ ID NO: 27, and a light chain having an amino acid sequence shown in SEQ ID NO: 13; or
(m) a heavy chain having an amino acid sequence shown in SEQ ID NO: 28, and a light chain having an amino acid sequence shown in SEQ ID NO: 13.

18. The bispecific antibody according to claim 14, wherein the bispecific antibody comprises a heavy chain having an amino acid sequence shown in SEQ ID NO: 31, and a light chain having an amino acid sequence shown in SEQ ID NO: 30.

19. An immunoconjugate, a pharmaceutically acceptable salt thereof, or a solvate thereof, comprising a single domain antibody according to claim 1, a heavy chain antibody comprising the single domain antibody, or a bispecific antibody comprising the single domain antibody; and an active moiety; optionally, the active moiety is selected from a group consisting of a toxin, a peptide tag, sortag, a radionuclide, a near-infrared fluorochromes, and a nanoparticle.

20. (canceled)

21. A pharmaceutical composition comprising a single domain antibody according to claim 1, a heavy chain antibody comprising the single domain antibody, a bispecific antibody comprising the single domain antibody, or an immunoconjugate comprising the single domain antibody; and a pharmaceutically acceptable carrier.

22. A nucleic acid molecule encoding a single domain antibody according to claim 1, a heavy chain antibody comprising the single domain antibody, or a bispecific antibody, or an immunoconjugate comprising the single domain antibody.

23. (canceled)

24. (canceled)

25. A method for treatment or prevention of a cancer, acute or chronic infection, or aging-associated ailment, comprising administering to a subject in need thereof a therapeutically or prophylactically effective amount of a single domain antibody according to claim 1, a heavy chain antibody comprising the single domain antibody, a bispecific antibody comprising the single domain antibody, or an immunoconjugate comprising the single domain antibody.

26. A polypeptide comprising a single domain antibody according to claim 1 or a heavy chain antibody comprising the single domain antibody; and one or more amino acid residues covalently linked to the N terminus, C terminus or anywhere therebetween of the single domain antibody or the heavy chain antibody.
